(19) United States
(12) Patent Application Publication
(10) Pub. No.: US 2007/0238955 A1
(43) Pub. Date: Oct. 11, 2007

(54) SYSTEMS AND METHODS FOR GENERATING DATA USING ONE OR MORE ENDOSCOPIC MICROSCOPY TECHNIQUES

(75) Inventors: Guillermo J. Tearney, Cambridge, MA (US); Nicusor Iftimia, North Chelmsford, MA (US); Brett Eugene Bouma, Quincy, MA (US); Wang-Yuhl Oh, Cambridge, MA (US)

Correspondence Address: DORSEY & WHITNEY LLP;INTELLECTUAL PROPERTY DEPARTMENT, 250 PARK AVENUE, NEW YORK, NY, 10177, US

(73) Assignee: The General Hospital Corporation, Boston, MA (US)

(21) Appl. No.: 11/622,854

(22) Filed: Jan. 12, 2007

Related U.S. Application Data
(60) Provisional application No. 60/759,936, filed on Jan. 18, 2006.

(51) Int. Cl. A61B 5/05

(52) U.S. Cl. 600/407

(57) ABSTRACT

Exemplary systems and methods for imaging at least one portion of a sample can be provided. For example, according to one exemplary embodiment of such systems and methods, it is possible to receive at least one first electro-magnetic radiation from the sample and at least one second electro-magnetic radiation from a reference using at least one arrangement. Such arrangement and the reference can be provided in an endoscope enclosure. The image data associated with the portion can be generated as a function of the first and second electro-magnetic radiations. In another exemplary embodiment, an endoscope arrangement can be provided for imaging such portion of the sample. The endoscope arrangement can include at least one interferometric arrangement configured to receive at least one electro-magnetic radiation from the sample, and situated within and at one end of an endoscope enclosure of the endoscope arrangement. According to yet another exemplary embodiment, at least one first Linnik interferometric arrangement at least one second fiber arrangement being in optical communication with the at least one first arrangement can be provided. The second arrangement can be configured to transmit an electro-magnetic radiation to the first arrangement. The first arrangement can be configured to receive an additional electro-magnetic radiation from the sample which can be associated with the first electro-magnetic radiation. The first arrangement can be configured to forward at least one third electro-magnetic radiation which is associated with the at least one second electro-magnetic radiation to the at least one second arrangement.

### CROSS-REFERENCE TO RELATED APPLICATION(S)

This application is based upon and claims the benefit of priority from U.S. Patent Application Ser. No. 60/759,936, filed Jan. 18, 2006, the entire disclosure of which is incorporated herein by reference.

### STATEMENT REGARDING FEDERALLY SPONSORED RESEARCH

The invention was made with the U.S. Government support under Contract No. BES-0086709 awarded by the National Science Foundation. Thus, the U.S. Government has certain rights in the invention.

### FIELD OF THE INVENTION

The present invention generally relates to systems and methods for generating data using one or more endoscopic microscopy techniques and, more particularly to e.g., generating such data using one or more high-resolution endoscopic microscopy techniques.

### BACKGROUND OF THE INVENTION

Medical imaging technology has advanced to provide physicians with important information regarding the macroscopic anatomy of patients. Imaging modalities such as radiography, magnetic resonance imaging, computed tomography, and ultrasound allow non-invasive investigations of large-scale structures in the human body with resolutions ranging from about 100 μm to 1 mm. However, for many disease processes, such as the early detection of cancer, a higher resolution may be desired in order to image subcellular nuclear features, which is important for performing an accurate diagnosis.

Two optical imaging techniques, e.g., optical coherence tomography (“OCT”) and confocal microscopy (“CM”), can provide microscopic non-invasive imaging of patients. While the OCT and CM systems and methods show potential for solving several important diagnostic problems, these techniques have certain technical requirements that can make endoscopic subcellular imaging difficult.

For example, the OCT methods and systems can provide a high axial resolution, but the OCT cross-sectional imaging provides a low transverse resolution in order to maintain a large depth of focus. Further, while the CM systems and methods can provide images in the human tissue with 1 μm transverse resolution, endoscopic implementation of CM may be difficult to achieve. Endoscopic CM systems, which generally use a small-diameter endoscopic probe, are difficult to implement due to certain endoscopic probe size constraints resulting from a requirement for a high numerical aperture (NA) objective lenses (NA≧0.7) and rapid beam scanning arrangements. In addition, since both OCT and CM methods and systems generally use lasers to illuminate a sample, the OCT and CM images likely contain significant coherent interference or speckle noise, which can degrade the resolution of the resulting images, e.g., by up to a factor of four.

One exemplary way to overcome certain limitations of the OCT and CM systems and methods, and provide true micron-resolution endoscopic imaging, is to combine the principles of these two technologies. Such resultant combined technology, at times referred to optical coherence microscopy (“OCM”), generally utilizes the high transverse resolution of CM and the high axial resolution of OCT. As a result, the exemplary OCM systems and methods are capable of providing a resolution on the order of 1 μm in all three dimensions. Furthermore, since optical sectioning in OCM does not necessitate a high numerical aperture (“NA”) lens, complexity and size of the focusing optics may be considerably reduced in comparison with other conventional systems and methods. However, similar to CM principles, the OCM systems and methods likely utilize a rapid scanning of a focused beam by way of a rapid beam scanning mechanism, and thus may also be difficult to implement in a small diameter endoscopic probe.

The OCM systems and methods can be implemented using a spatially incoherent illumination and parallel two-dimensional detection. This technology, known as full-field OCM (“FFOCM”) and also referred to as full-field optical coherence tomography (“FFOCT”), does not require a rapid beam scanning to form a microscopic image, and can significantly decrease speckle noise, while achieving the true resolution provided by the optical imaging system.

The above-described FFOCM systems and methods can facilitate a sub-micrometer imaging in human tissues. Such images can be obtained by acquiring several images, whereas each image may be acquired with a different position of the reference mirror. In this manner, for each mirror position, the interference between the reference and sample arms can be detected by a CCD camera for an entire image of the sample. Fringes may appear only when the reference and sample arms are matched to within the coherence length of light, which for a thermal light sources (e.g., conventional light bulb) may be in the sub-micrometer range. Mathematical manipulation of these images can allow for the generation of a high-resolution en-face image of structures that are provided deep within the tissue. The axial resolution for these images can be equivalent to the coherence length of the light source.

The FFOCM techniques generally combines the principles of OCT with the principles of CM to overcome certain disadvantages of each of these techniques. Exemplary advantages of FFOCM systems and methods over the conventional OCT systems and methods include, e.g., the ability to use inexpensive white light sources (e.g., light bulbs, lamps, and other thermal sources) to provide ultrahigh-resolution (sub-micrometer) imaging. The inherent broad bandwidth of these sources can enable imaging with an axial resolution of less than 1.0 μm. Furthermore, due to the spatial incoherence of the light source, speckle noise (which is commonly associated with coherent imaging techniques) can be significantly reduced. The reduction of speckle noise can greatly increase the diagnostic capability of the FFOCM techniques as compared to those of OCT.

Referring now to FIG. 1, a conventional full-field optical coherence microscopy (“FFOCM”) system 10 is arranged as a Linnik interferometer. The FFOCM system 10 shown in FIG. 1 includes a light detector (e.g., a CCD camera 12), a lens 14, a light source 16, a lens 18, and a partially reflecting mirror 20. This FFOCM system 10 also includes a reference arm 30 and a sample arm 32. The reference arm 30 can include a lens 22 and a reference mirror 24. The sample arm 32 can include a lens 26. In certain exemplary arrangements, the FFOCM system 10 can utilize an extended (e.g., multi-mode) light source 16 (e.g., a filament light source, also referred to herein as a thermal light source). In operation, the sample arm 32 transmits light toward a sample. The CCD camera 12 can receive light from the reference arm 30 and from the sample arm 32.

The various light paths implemented (using the conventional FFOCM system 10) as solid lines are free space light paths. It may be difficult to miniaturize the components of FIG. 1 within the confines of an endoscopic probe having a small diameter, e.g., less than 5 mm.

Additional advantages of the FFOCM systems and methods over those which utilize confocal microscopy (“CM”) techniques can include an ability to achieve submicron-level imaging without requiring a high numerical aperture objective lens. In combination with a low power (e.g., 10×, NA=0.4) microscope objective, FFOCM systems and methods may be capable of imaging human tissue with a transverse resolution similar to those which utilize such techniques, but without the need for a high numerical aperture objective. Also, the FFOCM systems and methods obtain an image without beam scanning, and is therefore, significantly simpler to implement.

The above-described properties of the FFOCM techniques, systems and methods suggest the possibility of use thereof for endoscopic cellular imaging in vivo. However, due to the complexities of miniaturizing the FFOCM system, it has been difficult to realize an endoscopic FFOCM system, which requires a small probe diameter.

Accordingly, it may be beneficial to address and/or overcome at least some of the deficiencies described herein above.

### OBJECTS AND SUMMARY OF THE INVENTION

One of the objectives of the present invention is to overcome certain deficiencies and shortcomings of the prior art systems and methods (including those described herein above), and provide exemplary embodiments of systems and methods for generating data using one or more endoscopic microscopy techniques and, more particularly to e.g., generating such data using one or more high-resolution endoscopic microscopy techniques.

According to one exemplary embodiment of the systems and methods of the present invention, exemplary systems and methods for imaging at least one portion of a sample can be provided. For example, according to one exemplary embodiment of such systems and methods, it is possible to receive at least one first electro-magnetic radiation from the sample and at least one second electro-magnetic radiation from a reference using at least one first arrangement. Such arrangement and the reference can be provided in an endoscope enclosure. The image data associated with the portion can be generated (e.g., using at least one second arrangement) as a function of the first and second electro-magnetic radiations.

For example, at least one third arrangement can be provided that may be in communication with the first arrangement and configured to receive at least one third electro-magnetic radiation from a further reference. The third arrangement can be provided outside of an endoscope enclosure. The further reference can be a translatable reference, and the third arrangement can be further configured to receive at least one fourth electro-magnetic radiation from a stationary reference. The translatable and stationary references can be provided externally from the endoscope enclosure. A fourth arrangement (e.g., a piezo-electric transducer) can be provided which is configured to move the translatable reference. The first arrangement can communicate with the third arrangement via a fiber arrangement (e.g., a single fiber and/or a plurality of fibers). The fiber arrangement can be a single model arrangement and/or a multi-mode arrangement. A first fiber of the fiber arrangement can be configured to transmit an electro-magnetic radiation to the sample, and the first fiber and a second fiber of the fiber arrangement may be configured to receive the first electro-magnetic radiation from the sample and the second electro-magnetic radiation from the reference. The first and second fibers can transmit a further electro-magnetic radiation for performing a dual balance detection.

According to one exemplary embodiment of the present invention, the further reference can be fixed, and the third arrangement can comprises a beam splitting arrangement providing a fourth electro-magnetic radiation and a fifth electro-magnetic radiation that are out of phases from one another. At least one fourth arrangement can be provided which may selectively forward the fourth and/or fifth electro-magnetic radiations to the first arrangement. The at least one fourth arrangement may be an optical switch.

In another exemplary embodiment of the present invention, the first arrangement can be an interferometric arrangement. Such interferometric arrangement can comprise a Michelson interferometer, a Linnik interferometer, a Mach-Zehnder interferometer, a common path interferometer, a Sagnac interferometer and/or a Mirau interferometer. Further, the interferometric arrangement may be monolithic. In another exemplary variant, the reference can include an attenuator and/or may be translatable.

In yet another exemplary embodiment, an endoscope arrangement can be provided for imaging such portion of the sample. The endoscope arrangement can include at least one interferometric arrangement configured to receive at least one electro-magnetic radiation from the sample, and situated within and at one end of an endoscope enclosure of the endoscope arrangement. For example, the one end of the endoscope enclosure can be provided in a proximity of the sample. The interferometric arrangement may be a Linnik interferometric arrangement. Such interferometric arrangement may be immersed in a fluid, and/or can comprise a beam splitting arrangement capable of providing a first further electro-magnetic radiation and a second further electro-magnetic radiation that are out of phases from one another. At least one further arrangement can be provided which may selectively forward the first and/or second further electro-magnetic radiations to at least one fiber arrangement. The third arrangement can be an optical switch and/or a plurality of fibers.

According to yet another exemplary embodiment, at least one first Linnik interferometric arrangement at least one second fiber arrangement being in optical communication with the at least one first arrangement can be provided. The second arrangement can be configured to transmit an electro-magnetic radiation to the first arrangement. The first arrangement can be configured to receive an additional electro-magnetic radiation from the sample which can be associated with the first electro-magnetic radiation. The first arrangement can be configured to forward at least one third electro-magnetic radiation which is associated with the at least one second electro-magnetic radiation to the at least one second arrangement.

According to a further variant of this exemplary embodiment, the second arrangement can be configured to transmit imaged data associated with the portion, and/or may be a fiber bundle. The third arrangement can be configured to receive the image data, and generate at least one image of the portion based on the image data. At least one first fiber of the second arrangement can be configured to transmit the first electro-magnetic radiation, and at least one second fiber of the at least one second arrangement may be configured to transmit the third electro-magnetic radiation. Further, at least one fiber of the second arrangement can be configured to transmit the first electro-magnetic radiation and the third electro-magnetic radiation.

In another exemplary variant, the first and second arrangements may be provided in a catheter enclosure or in an endoscope enclosure. The interferometric arrangement may be immersed in a fluid. The first arrangement can comprise a beam splitting arrangement which may provide the third electro-magnetic radiation and a fourth electro-magnetic radiation that are out of phases from one another. At least one third arrangement can be provided which can selectively forward the third and/or fourth further electro-magnetic radiations to the second arrangement. The third arrangement can be an optical switch and/or a plurality of fibers.

In another exemplary embodiment of the present invention, a method and system can be provided for performing endoscopic full-field optical coherence microscopy (“E-FFOCM”). Certain variants of the exemplary embodiments of the present invention can utilize an endoscopic probe having a fiber-optic bundle arranged in a Linnik interferometer, which can provide light to the endoscopic probe. The fiber-optic bundle can be single- or multi-mode, but preferably multimode for optimal coupling of the source light and detection of light remitted by the sample. By allowing light delivery through the fiber-optic bundle, the system can facilitate use of the E-FFOCM techniques in a catheter or endoscope. This exemplary embodiment can therefore enables, e.g., a high-resolution microscopy of surfaces of the body accessible by endoscope.

This exemplary configuration can be difficult to implement, since self-spatial coherence between sample and reference arms can be lost. Furthermore, a polarization may not be easily matched on a pixel-per-pixel basis. As a result, the interference contrast may be negligible, and it would be difficult to utilize coherence gating to obtain information at a depth within a sample.

According to yet another exemplary embodiment of the present invention, an optical-fiber imaging bundle can be used in both the sample and in the reference arms, which should be substantially identical in order to provide spatial and temporal coherence. Even though this exemplary configuration can reduce the spatial coherence mismatch in spatial modes between the arms, the sample arm fiber-optic bundle may change with respect to the reference arm fiber-optic bundle during the diagnostic procedure. As a result, the reference and sample arms can both be spatially and temporally mismatched, possibly preventing a desired level of interference.

In still another exemplary embodiment of the present invention, to further improve the temporal and spatial coherence match between reference and sample arms, one fiber-optic bundle can be used to transmit and/or receive the both reference and sample arm light. In such exemplary embodiment, the interferometer can be placed distal to the fiber-optic bundle. The reference arm and sample arm illumination light can travel through the same bundle. At the distal end of the endoscope, the reference arm path can be incident on a mirror mounted to a small linear translator such as a piezoelectric stack. The sample and reference arm light may be combined at the distal beam splitter and transmitted back through the fiber bundle. Since the sample and reference arm paths can traverse the same bundle, they generally remain spatially and temporally coherent with respect to each other, thus facilitating a high contrast interference at the CCD. Furthermore, a dispersion mismatch caused by the bundle can be balanced due to the common paths of the reference and sample arms.

In accordance with still another exemplary embodiment of the present invention, a system for endoscopic imaging can include a fiber-optic bundle and an endoscopic probe which may be coupled to the fiber-optic bundle. In the exemplary variants of this exemplary embodiment, the endoscopic probe can include an interferometer reference arm and an interferometer sample arm. In other exemplary variants, the interferometer reference arm can include a linear actuator and a mirror coupled to the linear actuator. In further exemplary variants, the system for endoscopic imaging may further include a light source interferometer having a light source interferometer reference arm and a light source interferometer sample arm. A light source interferometer reference arm can include a linear actuator and a mirror coupled to the linear actuator.

Other features and advantages of the present invention will become apparent upon reading the following detailed description of embodiments of the invention, when taken in conjunction with the appended claims.

### BRIEF DESCRIPTION OF THE DRAWINGS

Further objects, features and advantages of the present invention will become apparent from the following detailed description taken in conjunction with the accompanying figures showing illustrative embodiments of the present invention, in which:

FIG. 1 is a schematic diagram of a conventional free-space full-field optical coherence microscopy (“FFOCM”) system arranged as a Linnik interferometer;

FIG. 2 is a schematic diagram of a Linnik interferometer having a fiber bundle;

FIG. 3 is a schematic diagram of a Linnik interferometer having a fiber bundle in both a reference and a sample arm;

FIG. 4 is a schematic diagram of an exemplary embodiment of an endoscopic full-field optical coherence microscopy (“E-FFOCM”) system having a single fiber bundle used in both the reference and sample arms, whereas a Linnik interferometer is placed at the distal end of the E-FFOCM system, e.g., in an endoscopic probe;

FIG. 5 is a schematic diagram of another exemplary embodiment of the E-FFOCM system shown in FIG. 4;

FIG. 6A is a schematic diagram of a distal end of an endoscopic probe assembly, having a single fiber bundle used both for illumination and detection, which can be used in the exemplary E-FFOCM system shown in FIG. 4;

FIG. 6B is a schematic diagram of another exemplary embodiment of an endoscopic probe assembly, having two fiber bundles used separately for illumination and detection, which can be used in the exemplary E-FFOCM system shown in FIG. 3;

FIG. 7A is a schematic diagram of an exemplary embodiment of an endoscopic probe assembly having single lens distal optics, which can be used in the exemplary embodiment of the E-FFOCM system;

FIG. 7B is a schematic diagram of an exemplary embodiment of an endoscopic probe assembly having dual-lens distal optics, which can be used in the exemplary embodiment of the E-FFOCM system;

FIG. 8 is a schematic diagram of another exemplary embodiment of the endoscopic probe assembly having a monolithic distal interferometer, which can be used in the exemplary embodiment of the E-FFOCM system;

FIG. 9A is a schematic diagram of a further exemplary embodiment of the endoscopic probe assembly having monolithic distal optics and having an attenuator in the reference arm, which can be used in the exemplary embodiment of the E-FFOCM system;

FIG. 9B is a schematic diagram of yet another exemplary embodiment of the endoscopic probe assembly having monolithic distal optics and having an attenuator in the reference arm, which can be used in the exemplary embodiment of the E-FFOCM system;

FIG. 10 is a schematic diagram of another exemplary embodiment of an endoscopic probe assembly having optics in a Mirau configuration, which can be used in the exemplary embodiment of the E-FFOCM system;

FIG. 11 is a schematic diagram of a further exemplary embodiment of an endoscopic probe assembly having dual balanced detection with two fiber bundles, which can be used in the exemplary embodiment of the E-FFOCM system;

FIG. 12 is a schematic diagram of an exemplary embodiment of a spectral domain E-FFOCM system;

FIG. 13 is a schematic diagram of a further exemplary embodiment of the E-FFOCM system;

FIG. 14 is a schematic diagram of still another exemplary embodiment of the E-FFOCM system having a light source interferometer and including the endoscopic probe as in FIG. 6A;

FIG. 15A is a schematic diagram of a particular exemplary embodiment of the endoscopic probe assembly having a side-looking probe configuration, which can be used in the exemplary embodiment of the E-FFOCM system;

FIG. 15B is a schematic diagram of a further exemplary embodiment of the endoscopic probe assembly having a forward-looking probe configuration, which can be used in the exemplary embodiment of the E-FFOCM system;

FIG. 16A is a schematic diagram of a first exemplary embodiment of a monolithic endoscopic probe assembly having a side-looking probe configuration having a stationary mirror, and using one optical fiber, which can be used in the exemplary embodiment of the E-FFOCM system;

FIG. 16B is a schematic diagram of a second exemplary embodiment of the monolithic endoscopic probe assembly having a forward-looking probe configuration having a stationary mirror, and using one optical fiber, which can be used in the exemplary embodiment of the E-FFOCM system;

FIG. 16C is a schematic diagram of a third exemplary embodiment of the monolithic endoscopic probe assembly having a forward-looking probe configuration having a stationary mirror, and that uses two optical fibers for illumination and detection, respectively, which can be used in the exemplary embodiment of the E-FFOCM system;

FIG. 16D is a schematic diagram of a fourth exemplary embodiment of the monolithic endoscopic probe assembly having a side-looking probe configuration having a stationary mirror, and that can use two optical fibers for illumination and detection respectively, which can be used in the exemplary embodiment of the E-FFOCM system;

FIG. 17 is a schematic diagram of an exemplary embodiment of a light source interferometer providing reflected and transmitted light to and from an interferometer via a 2′ 1 switch, which can be used in the exemplary embodiment of the E-FFOCM system shown, for example, in FIG. 14;

FIG. 18 is a schematic diagram of another exemplary embodiment of the light source interferometer providing a polarization modulation, which can be used in the exemplary embodiment of the E-FFOCM system shown, for example, in FIG. 14;

FIG. 19 is a schematic diagram of still another exemplary embodiment of the light source interferometer having a coherent light source with a single mode fiber interferometer and a multi-mode illumination fiber bundle, which can be used in the exemplary embodiment of the E-FFOCM system shown, for example, in FIG. 14;

FIG. 20 is a schematic diagram of an exemplary embodiment of the E-FFOCM system having a light source interferometer with a movable mirror for scanning, in which three-dimensional volumetric imaging can be obtained without any mechanical scanning in the endoscopic probe;

FIG. 21A is a schematic diagram of a first exemplary embodiment of the endoscopic probe assembly including line-scan imaging with a movable beam splitter, that provides a scan in a transverse direction, and which can be used in the exemplary embodiment of the E-FFOCM system;

FIG. 21B is a schematic diagram of a second exemplary embodiment of the endoscopic probe assembly having a side-looking configuration, and that includes line-scan imaging with a movable distal interferometer, which provides a scan in a transverse direction, and which can be used in the exemplary embodiment of the E-FFOCM system;

FIG. 21C is a schematic diagram of a third exemplary embodiment of the endoscopic probe assembly having a forward-looking configuration, that includes line-scan imaging with a movable distal interferometer, which provides a scan in transverse direction, and which can be used in the exemplary embodiment of the E-FFOCM system;

FIG. 22A is a schematic diagram of a first exemplary embodiment of the endoscopic probe assembly having a mechanical scanning arrangement;

FIG. 22B is a schematic diagram of a second exemplary embodiment of the endoscopic probe assembly having the mechanical scanning arrangement;

FIG. 22C is a schematic diagram of a third exemplary embodiment of the endoscopic probe assembly having the mechanical scanning arrangement;

FIG. 22D is a schematic diagram of a fourth exemplary embodiment of the endoscopic probe assembly having the mechanical scanning arrangement;

FIG. 23A is an exemplary image generated using the exemplary E-FFOCM system shown in FIG. 13 for which a piezoelectric (PZT) linear translator is turned off;

FIG. 23B is an exemplary image generated using the exemplary E-FFOCM system of FIG. 13 for which the PZT is turned on; and

FIG. 24 is an image generated using an FFOCM system with Michelson source interferometer showing an en face sectional image of the African frog tadpole Xenopus laevis, obtained ex vivo, 200 mm below the surface.

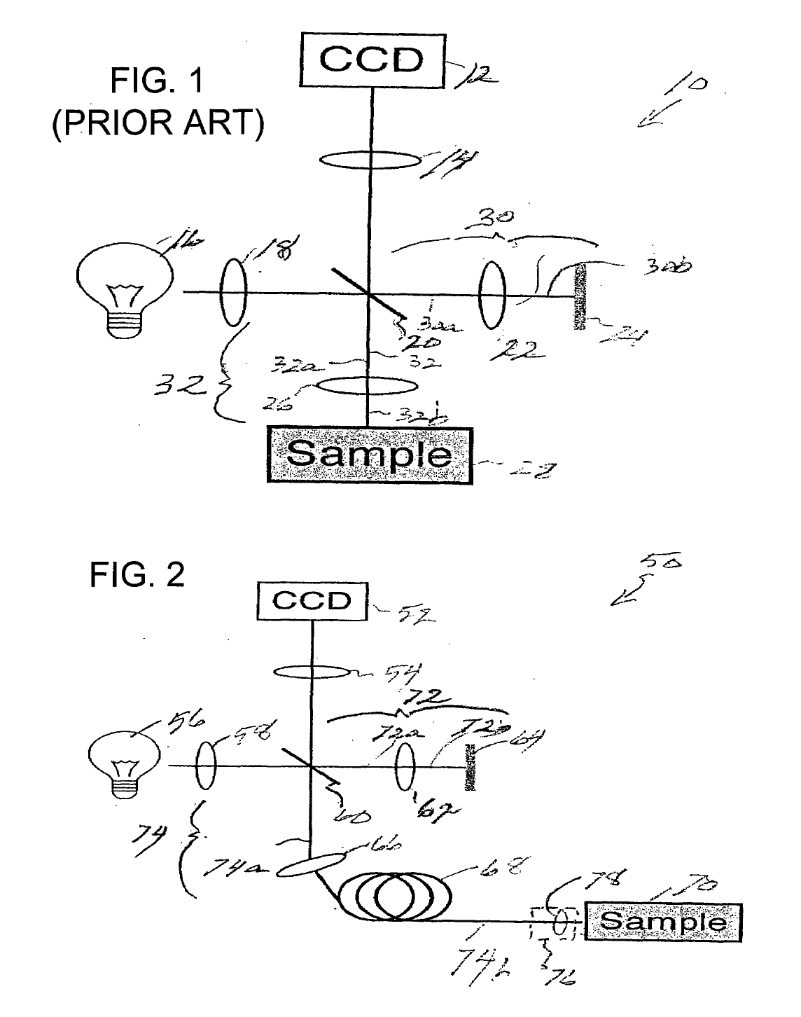

Throughout the figures, the same reference numerals and characters, unless otherwise stated, are used to denote like features, elements, components or portions of the illustrated embodiments. Moreover, while the subject invention will now be described in detail with reference to the figures, it is done so in connection with the illustrative embodiments. It is intended that changes and modifications can be made to the described embodiments without departing from the true scope and spirit of the subject invention as defined by the appended claims.

### DETAILED DESCRIPTION OF EXEMPLARY EMBODIMENTS

Prior to providing a detailed description of the various exemplary embodiments of the methods and systems for endoscopic microscopy according to the present invention, some introductory concepts and terminology are provided below. As used herein, the term “endoscopic probe” can be used to describe one or more portions of an exemplary embodiment of an endoscopic system, which can be inserted into a human or animal body in order to obtain an image of tissue within the body.

As used herein, the term “monolithic” can be used to describe a structure formed as a single piece, which may have more than one optical function. As used herein, the term “hybrid” can be used to describe a structure formed as a plurality of pieces, each piece having one optical function.

The exemplary embodiments of the methods and systems according to the present invention described below can be used with any wavelength of light or electro-magnetic radiation, including but not limited to visible light and near infrared light.

Referring FIG. 2, an exemplary embodiment of an endoscopic full-field optical coherence microscopy (“E-FFOCM”) system 50 according to the present invention can include a light detector, for example, a charge coupled device (“CCD”) camera 52, a lens 54, a light source 56, a lens 58, and a partially reflecting mirror 60. The E-FFOCM system 50 also includes a reference arm 72 and a sample arm 74. The reference arm 70 can include a lens 62 and a reference mirror 64. The sample arm 74 can include a lens 66, a fiber bundle 68, and a lens 78. In certain exemplary embodiments of the present invention, the lens 78 can be provided within an endoscopic probe 76, facilitating E-FFOCM. Certain exemplary embodiments for which the lens 78 is not facilitated within the endoscopic probe 76 can provide full-field optical coherence microscopy (“FFOCM”).

The exemplary embodiment of the E-FFOCM system 50 can operate with the fiber-optic imaging bundle 68 to convey light from a light source 56 to a sample 70. The fiber-optic bundle 68 can also receive an image from the sample 70, and convey the image back to the light detector 52. The image from the sample arm 74 can then interfere with light from the reference arm 72 within the light detector 52, e.g., within the CCD camera 52. The fiber-optic bundle 68 can operate in single or multi-mode, and preferably in multimode, since multimode operation may provide a preferable coupling of the source light and the received light remitted by the sample 70.

In this exemplary configuration of the exemplary embodiment of the present invention shown in FIG. 2, self-spatial coherence between the sample arm 74 and reference arm 72 may not be sufficient to provide high resolution images. Furthermore, polarization would be poorly matched on a pixel-per-pixel basis. As a result, interference contrast would be low and coherence gating could not be well utilized to obtain a quality image at an appreciable depth within the sample.

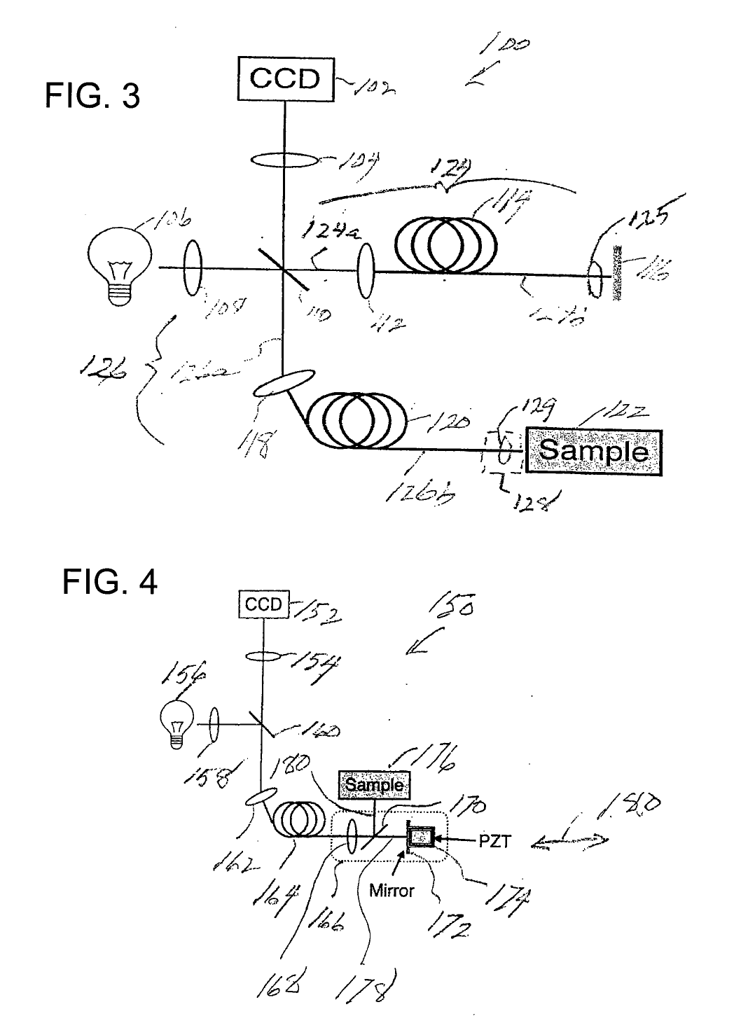

FIG. 3 shows another exemplary embodiment of the E-FFOCM system 100 which can include a light detector, for example, a CCD camera 102, a lens 104, a light source 106, a lens 108, and a partially reflecting mirror 110. The exemplary E-FFOCM system 100 can also include a reference arm 124 and a sample arm 126. The reference arm 124 can include a lens 112, a first fiber-optic bundle 114, and a reference mirror 116. The sample arm 126 can include a lens 118 and a second fiber-optic bundle 120, which may be similar to the first fiber-optic bundle 114, and a lens 129. In certain exemplary embodiments, the lens 129 can be facilitated within an endoscopic probe 128, and provide E-FFOCM. Exemplary embodiments for which the lens 129 is not situated within the endoscopic probe can provide full-field optical coherence microscopy (“FFOCM”).

It may be difficult to match spatial and temporal coherence between two fiber-optic bundles 114, 120, since they would may be very similar or approximately identical. While this exemplary arrangement can minimize the above-described spatial coherence mismatch in spatial modes between the two arms 124, 126 if the two fiber-optic bundles 114, 120 are initially matched, it is likely that the sample arm fiber-optic bundle 120 may change with respect to the reference arm bundle 114 during the diagnostic procedure. As a result, the reference and sample arms 114, 120, respectively, may be less-than-optimally spatially and temporally matched, thus possibly preventing or reducing the desired interference at the CCD camera 102.

FIG. 4 shows another exemplary embodiment of the E-FFOCM system 150 which can include a light detector, for example, a CCD camera 152, a lens 154, a light source 156, a lens 158, and a partially reflecting mirror 160. The E-FFOCM system 150 can also include a lens 162, a fiber-optic bundle 164, and an endoscopic probe 166. The probe 166 can include a lens 168, another partially reflecting mirror 170, and a reference mirror 172. The probe 166 includes a reference arm 178 and a sample arm 180. The probe 166 can also a linear actuator, for example, a piezoelectric (PZT) stack 174, coupled to the reference mirror 172. The sample arm 180 can transmit light toward a sample 176.

The exemplary E-FFOCM system 150 of FIG. 4 may include the fiber-optic bundle 164 and a distal end, e.g., also can be referred to herein as an endoscopic probe 166. The probe 166 can include the interferometer having the lens 168, the partially reflecting mirror 170, and the mirror 172 coupled to the linear actuator 174. In operation, the linear actuator 174 can move the mirror 172 along an axis 180.

During such operation, the one fiber-optic bundle 164 can be used to both transmit light and receive light. The light from both the reference and the sample arms 178, 180 can travel through the same fiber-optic bundle 164. This exemplary embodiment according to the present invention may address the above-described temporal and spatial coherence potential mismatches between reference and sample arms 178, 180, respectively. In this exemplary arrangement, the interferometer can be placed distal with respect to the fiber-optic bundle 164, within the probe 166.

At the distal end of the endoscopic probe 166, the light generated by the light source 156 and passing through the partially reflective mirror 170 (e.g., also may be referred to as a beam splitter) can be incident upon the mirror 172, thus forming the reference arm 178. The light generated by the light source 156 and reflecting from the partially reflecting mirror 170 can also be incident upon the sample 176, thus forming the sample arm 180. The returning light from the sample arm and returning light from the reference arm can be combined at the partially reflective mirror and transmitted back through the fiber bundle 164. Since the sample and reference arm paths may traverse the same fiber-optic bundle 164, they can remain spatially and temporally coherent with respect to each other, thus facilitating a high contrast interference at the CCD light detector 152. Furthermore, the dispersion mismatch in the fiber-optic bundle 164 can likewise be balanced due to the common paths through the same fiber-optic bundle 164.

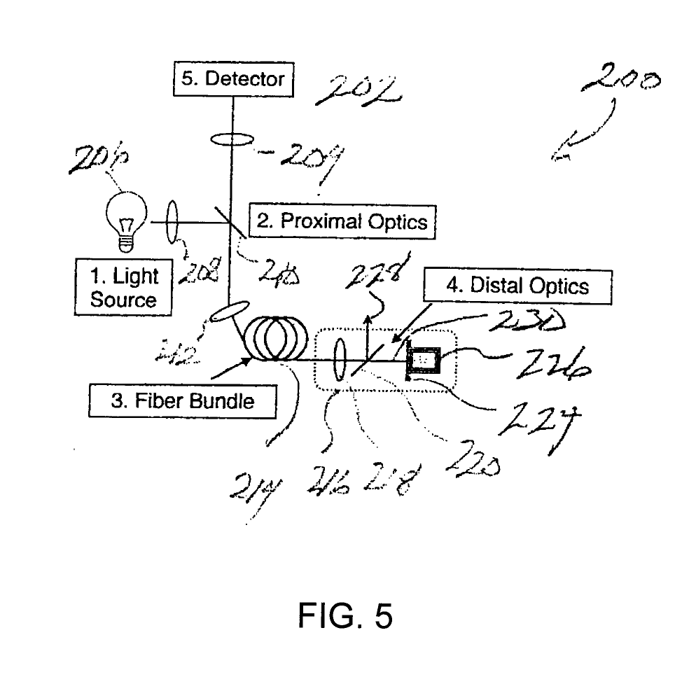

FIG. 5 shows another exemplary embodiment of the E-FFOCM system 200 according to the present invention which can include a light detector, for example, a CCD camera 202, a lens 204, a light source 206, a lens 208, and a partially reflecting mirror 210. The exemplary E-FFOCM system 200 can also include a lens 212, a fiber-optic bundle 214, and a probe 216. The probe 216 can include a lens 218, another partially reflecting mirror 220, and a reference mirror 224. The E-FFOCM system 200 includes a reference arm 230 and a sample arm 228. The probe 218 can also include a linear actuator, for example, a piezoelectric (PZT) stack 226, coupled to the reference mirror. The sample arm 228 can transmit light toward s sample (not shown).

Contrary to certain imaging technologies that may require a high-brightness or spatially coherent source, the light source 206 can be used with the exemplary E-FFOCM system 150 can be of a variety of types, including but not limited to, a broadband and an incoherent light source. Filament-based thermal light sources such as light bulbs, incandescent lamps, discharge lamps, etc. may be preferable since they can provide a high output power and very large spectral bandwidth, at a very low cost. Examples of this type of source may include Halogen, Tungsten, Xenon, and Mercury. Other spatially incoherent sources such as LED (light emitting diode), SLED (surface emitting LED), EELED (edge emitting LED), and multimode ASE, may also be utilized. In other exemplary embodiments, coherent light sources can be used, such as lasers. The coherent light sources generally have higher cost, and tend to result in images having a higher level of speckle noise.

The fiber-optic bundle 214 can be single-mode, but is preferably to us multi-mode bundle. Alternatively, the fiber-optic bundle 214 can be comprised of one or more separate optical fibers, which can each be single mode, and preferably multi-mode for optimal coupling efficiency.

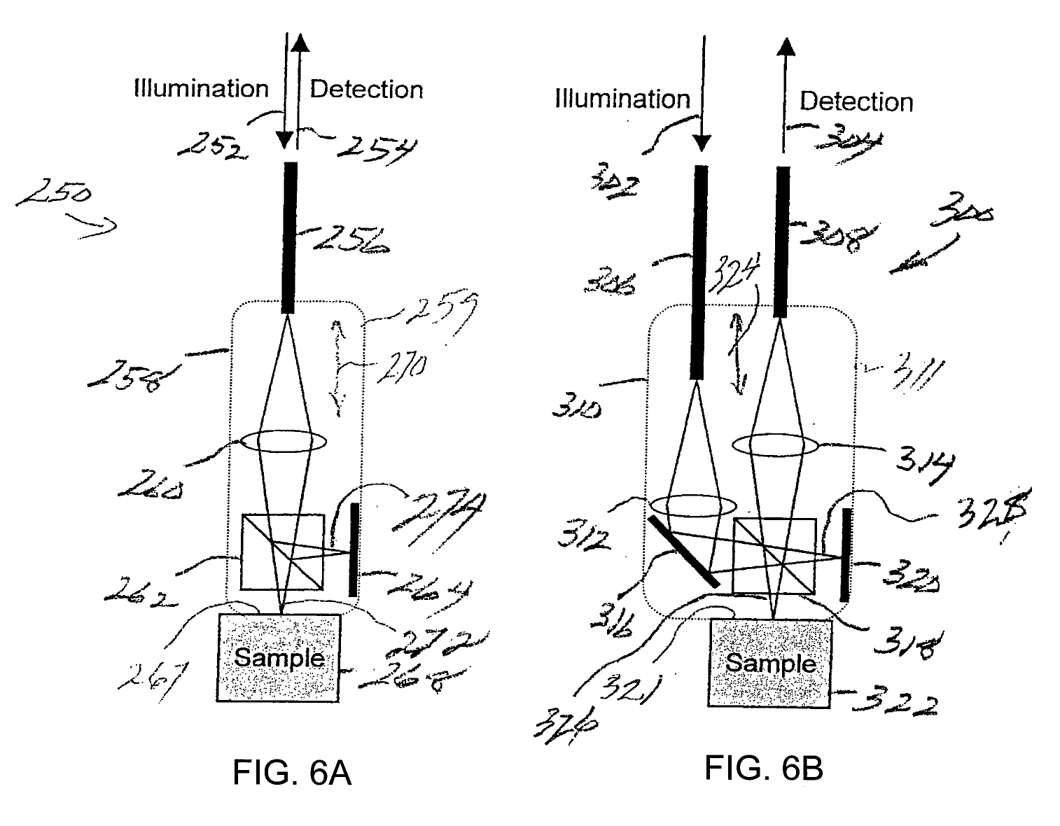

FIG. 6A shows an exemplary embodiment of a forward-looking endoscopic probe assembly 250 can be used with the single fiber-optic bundle arrangement 256 shown in FIG. 4. The exemplary probe assembly 250 can include a probe 258 having a sheath 259 with a window 267, a fixed lens 260, a cube-type beam splitter 262, and a mirror 264. The probe 258 may be coupled to a fiber-optic bundle 256. In this exemplary configuration, a sample arm 272 may be disposed along an axis 270 of the probe 258, and a reference arm 274 may be disposed perpendicular to the axis 270 of the probe 258.

In operation, an illumination light 252 may split at the cube-type beam splitter 262, and impinge upon both a sample 268 and the mirror 264. The light, which may impinge upon the mirror 264, can form the reference arm 274, and the light, which may impinge upon the sample 268, can form the sample arm 272. The light from both the reference arm and the sample arm 274, 272 can return as a detection light 254 via the fiber-optic bundle 256.

FIG. 6B shows another exemplary embodiment of the forward-looking endoscopic probe assembly 300 according to the present invention which can be used with a two fiber-optic bundle arrangement 306, 308 shown in FIG. 3. The exemplary probe assembly 300 can include a probe 310 having a sheath 311 with a window 321, a fixed lens 314, another fixed lens 316, a cube-type beam splitter 318, a mirror 320, and another mirror 316. The probe 310 may be coupled to a first fiber-optic bundle 308 and a second fiber-optic bundle 306. A sample arm 326 can be disposed along an axis 324 of the probe 310, and a reference arm 328 can be disposed perpendicular to the axis 324 of the probe 310.

The exemplary forward-looking endoscopic probe assembly 300 can be used with the two fiber-optic bundles shown in FIG. 3. The lenses of FIG. 3 may be disposed within the probe 310.

In operation, the illumination light can impinge upon the mirror 316, split at the cube-type beam splitter 318, and impinge upon both the sample 322 and upon the mirror 320. The light, which can impinges upon the mirror 320, may form the reference arm 328, and the light, which can impinge upon the sample 322, may forms the sample arm 326. The light from both the reference arm and the sample arm 328, 326 can return as the detection light 304 to the second fiber-optic bundle 308.

Certain exemplary embodiments according to the present invention, which use the probe assemblies 250, 300 of FIGS. 6A and 6B, can use a wavelength-swept light source to provide optical frequency domain imaging (“OFDI”), which can be referred to Fourier domain OCT with a wavelength-swept light source. In this exemplary arrangement, images from different depth positions within the sample may be generated by Fourier transforming the signals received by a two-dimensional detector array (e.g., area scan camera) without need of a moving reference mirror. The wavelength scanning frequency of the light source can be matched to the frame rate of the detector array.

In certain embodiments of OFDI, a wavelength-swept laser can be used as a light source, and total lasing bandwidth may not be broad enough to provide cellular level axial resolution. Additionally, using a laser light source may result in an increased speckle noise because of its coherency.

Other exemplary embodiments of OFDI can instead use a broadband light source with a wavelength scanning filter. Since the confocal length of the objective lens (e.g., element 260 of FIG. 6A) may be only a few tens of micrometers, it may be necessary for such arrangement to use a wavelength scanning filter having a relatively broad band, utilizing several wavelength components in a wavelength tuning cycle. In certain exemplary embodiments, a Lyot filter may be used as the scanning filter. The wavelength scanning filter can be either a bandpass type filter or a filter with a sinusoidal transmission profile. In other exemplary embodiments, the wavelength scanning filter can be placed in front of the detector array.

The Fourier domain OCT (“OFDI”) arrangement can also be implemented using a detector array with large number of imaging pixels. By directing several different wavelengths onto the different sections of the large area detector array, the imaging light can be wavelength multiplexed across the detector array. Signals detected at each array detector area, which may correspond to a distinct wavelength, can be Fourier-transformed to construct en-face images for different depth positions with the sample. This exemplary technique may provide an advantageous imaging speed since a single frame of the large area array detector can be used to obtain several en-face images associated with several different depth positions, respectively.

In certain exemplary arrangements for which illumination light source can be coupled to an imaging fiber-optic bundle, proximal optics may direct the illumination light into the fiber-optic bundle and allow light, which returns to the fiber-optic bundle from a sample, to be directed to a detector array. In the exemplary arrangements for which the illumination light source may be separated from the imaging fiber-optic bundle, the proximal optics can likely only image the proximal end of the fiber-optic bundle on the detector array.

The fiber-optic bundles 306, 308 may contain one or more fibers, and preferably include enough fibers to transmit image data. The fibers may be single-mode or multi-mode, and are preferably multi-mode to increase the detection of light from the sample and to decrease the contribution of speckle noise in the final image. The entire bundle may be of fused or leached type, depending on the application.

The distal optics lens or lenses, located in the exemplary endoscopic probe, (e.g., elements 312, 314 of FIG. 6A) can provide a lateral resolution in accordance with a desired application. Contrary to confocal microscopy, this lens is generally not utilized to achieve optical sectioning in tissue, and therefore, the axial resolution provided by the lens is not necessary. Table 1 shows the exemplary numerical apertures as a function of wavelength for two different axial spatial resolutions (e.g., 1 and 2 micrometers). For most wavelengths in the visible and near infrared, a numerical aperture of less than 0.5 may greatly decrease the complexity of the endoscope lens. These exemplary configurations are significantly different than confocal microscopy where numerical apertures greater than 0.7 are generally required for high-resolution imaging.

TABLE 1
Numerical apertures required for a 1 and 2 μm spatial
resolution (water immersion is assumed).

| Wavelength (μm) | NA 1 μm | NA 2 μm |
|---|---|---|
| 0.4 | 0.18 | 0.09 |
| 0.5 | 0.23 | 0.11 |
| 0.6 | 0.28 | 0.14 |
| 0.7 | 0.32 | 0.16 |
| 0.8 | 0.37 | 0.18 |
| 0.9 | 0.41 | 0.21 |
| 1 | 0.46 | 0.23 |
| 1.1 | 0.5 | 0.25 |
| 1.2 | 0.55 | 0.28 |
| 1.3 | 0.6 | 0.3 |
| 1.4 | 0.64 | 0.32 |
| 1.5 | 0.69 | 0.34 |
| 1.6 | 0.73 | 0.37 |
| 1.7 | 0.78 | 0.39 |
| 1.8 | 0.83 | 0.41 |
| 1.9 | 0.87 | 0.44 |
| 2 | 0.92 | 0.46 |

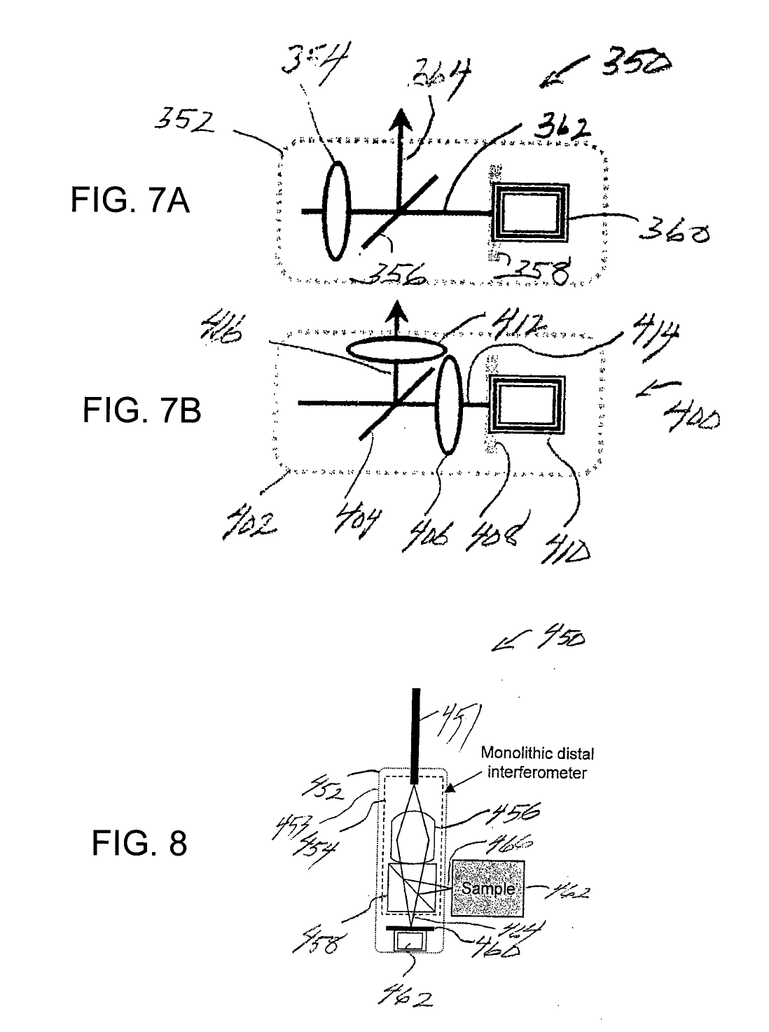

FIG. 7A shows an exemplary embodiment of a side-looking endoscopic probe 350 which can be used with the exemplary single fiber-optic bundle arrangement shown in FIG. 4. The probe 350 can include a sheath 352, a fixed lens 354, a partially reflective mirror 356, and a mirror 358 disposed on a linear actuator 360, e.g., a piezoelectric (PZT) stack. The probe 350 may include a reference arm 362 and a sample arm 364.

In this exemplary embodiment, the imaging lens 354 can be disposed before the distal interferometer. This exemplary configuration has the advantage that the same lens 354 is utilized for the reference and sample arm paths, thereby possibly reducing coherence, polarization, and dispersion imbalances between the reference and sample arms.

FIG. 7B shows another exemplary embodiment of the side-looking endoscopic probe 400, which can also be used with the exemplary single fiber-optic bundle arrangement shown in FIG. 4. The probe 400 can include a sheath 402, a partially reflective mirror 404, a fixed lens 406, and a mirror 408 disposed on a linear actuator 3410, e.g., a piezoelectric (PZT) stack. The probe 400 may also include another fixed lens 412. The probe 400 can include a reference arm 414 and a sample arm 416.

In the exemplary probe 400, two objective lenses 406, 412 may be utilized, e.g., one for the sample arm 416 and the other for the reference arm 414. This exemplary arrangement may be advantageous if the working distance for a single objective would be such that it cannot accommodate the interferometer. Two lenses 412, 406 of this exemplary arrangement can be selected to be sufficiently similar, i.e., matched, so as not to induce significant dispersion imbalances between the reference and sample arm paths 414, 416, respectively.

It may be desirable for the immersion index of the lens or lenses (e.g., element 354 or FIG. 7A; and elements 412 and 406 of FIG. 7B) to match that of human tissue (n=1.33−1.40). As a result, for optimal operation in tissues, in certain exemplary embodiments, the entire objective and distal optics can be immersed in a fluid, e.g., the sheaths 352, 402 of FIGS. 7A and 7B may be filled with a fluid, and the sheaths 352, 402 and the lenses 354, 412, 406 can be designed for diffraction-limited performance under conditions of immersion.

The interferometer can be of many configurations including Mach-Zehnder, Sagnac, and Michelson. In order to fit the interferometer within the endoscopic probes 350, 400 of FIGS. 7A and 7B, respectively, exemplary miniaturization techniques can be employed. According to certain exemplary embodiments, e.g., the exemplary configurations of FIGS. 6A and 6B, a cube-type beam splitter (e.g., elements 262, 318) can be used. In other exemplary arrangements, other beam splitters may be used, including but not limited to, partially reflecting mirrors 356 (of FIG. 7A) 404 (of FIG. 7B) and pellicle splitters. The beam splitters can have a wide range of splitting ratios, with a preferred ratio of 50:50. However, other exemplary ratios can range from 80:20 to 20:80.

FIG. 8 shows another exemplary embodiment of a side-looking endoscopic probe assembly 450 according to the present invention which can be used with the exemplary single fiber-optic bundle arrangement 451 shown in FIG. 4. The exemplary probe assembly 450 can include an endoscopic probe 452 having a sheath 452, and an interferometer 454. The interferometer 454 may include a fixed lens 456, and a cube-type beam splitter 458. The probe 452 can further include a mirror 460 disposed on a linear actuator 462, e.g., a piezoelectric (PZT) stack. The probe 452 may include a reference arm 464 and a sample arm 466, which can direct the light at a sample 462.

The interferometer 454 can be monolithic in order to reduce size. The monolithic structure can also reduce the deleterious effects of vibrational motion of the reference arm.

In certain exemplary embodiments, the reference mirror 460 can be a metal mirror. According to other exemplary embodiments, the reference mirror 460 can be a dielectric mirror, or a facet of an optical component used in the interferometer. In one exemplary embodiment, the reference mirror 460 may be a flat homogeneous medium and a reference reflection can arise from Fresnel reflection from a glass-water interface.

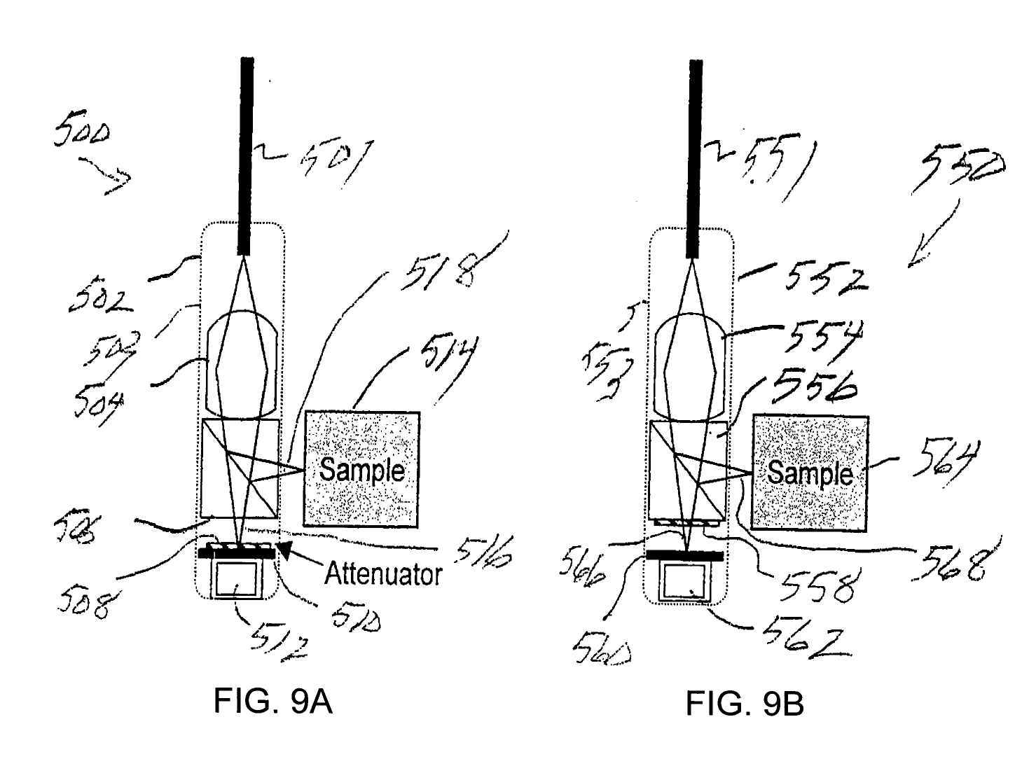

FIG. 9A shows an exemplary embodiment of the side-looking endoscopic probe assembly 500 according to the present invention which can be used with the exemplary single fiber-optic bundle arrangement 501 shown in FIG. 4. The exemplary probe assembly 500 can include an endoscopic probe 502 having a sheath 503, a fixed lens 504, a cube-type beam splitter 506, and a mirror 510 disposed on a linear actuator 512, e.g., a piezoelectric (PZT) stack. The probe 502 may include a reference arm 516 and a sample arm 518, which can direct light at a sample 514.

The probe 502 can also include an attenuator 508 disposed on or proximate to the mirror 510. The attenuator 508 may generally be disposed between the reference mirror 510 and the beam splitter 506. In one exemplary embodiment, the attenuator 508 can be coupled to the reference mirror 510. The attenuator 508 can be advantageous when reflected light in the reference arm has too high an intensity.

FIG. 9B shows another exemplary embodiment of the side-looking endoscopic probe assembly 550 which can be used with the exemplary single fiber-optic bundle arrangement 551 shown in FIG. 4. The probe assembly 550 can include an endoscopic probe 552 having a sheath 553, a fixed lens 554, a cube-type beam splitter 556, and a mirror 560 disposed on a linear actuator 562, e.g., a piezoelectric (PZT) stack. The probe 552 may include a reference arm 566 and a sample arm 568, which directs light at a sample 564.

The probe 552 can also include an attenuator 558 disposed on or proximate to the beam splitter 556. The attenuator 558 may generally be disposed between the reference mirror 560 and the beam splitter 556. In one exemplary embodiment, the attenuator 558 can be coupled to the beam splitter 556.

As described above, in certain exemplary embodiments, the reference mirror 560 can be coupled to a piezoelectric transducer (PZT) 562, which can provide linear translation of the reference mirror 560. Motion of the PZT 562 can be synchronized to a light detector, e.g., the light detector 202 of FIG. 5, so that different phase mismatches between reference and sample arms can be recorded in a synchronized manner. According to another exemplary embodiment, phase mismatches of 0, p/4, p/2, and 3p/2 can be provided.

The PZT (e.g., element 512 of FIG. 9A, and element 562 of FIG. 9B) can be driven with any modulation signal, e.g. sinusoidal, square, or triangle, and can provide linear translation accordingly. In exemplary embodiment of the arrangement, the PZT can provide quadrature modulation, e.g., four positions of the mirror 510 according to increments of p/2 wavelengths. While the modulation signal does not have to be smooth sinusoidal, higher order terms of modulation, which are close to a PZT resonant frequency should preferably be removed. The way for obtaining quadrature modulation is not limited to mechanical motion of the reference mirror. For example, other exemplary ways can include the use of electro-optic phase modulation, polarization modulation, etc., to obtain the quadrature modulation.

The image construction procedure is also not limited to quadrature modulation. Indeed, e.g., two phases with p phase mismatch, 5 phases, or different modulation schemes utilizing any number of phase sets can be used for image construction, as well as others.

As described above, the endoscopic probe (e.g., element 502 of FIG. 9A, and element 552 of FIG. 9B) can be enclosed in a transparent sheath (e.g., element 503 of FIG. 9A, and element 553 of FIG. 9B), or alternatively, an opaque sheath with a transparent window in the sample arm. In certain exemplary embodiments, the sheath or window itself may have an inner surface, which can form a reference reflector in place of the reference mirror 510, 552 of FIGS. 9A and 9B, respectively, when non-mechanical modulation is used for phase modulation. In this exemplary embodiment of the arrangement, all facets and interfaces on the interior of the endoscopic probe, which are supposed to be non-reflective, can be anti-refection coated to prevent any unnecessary reflection.

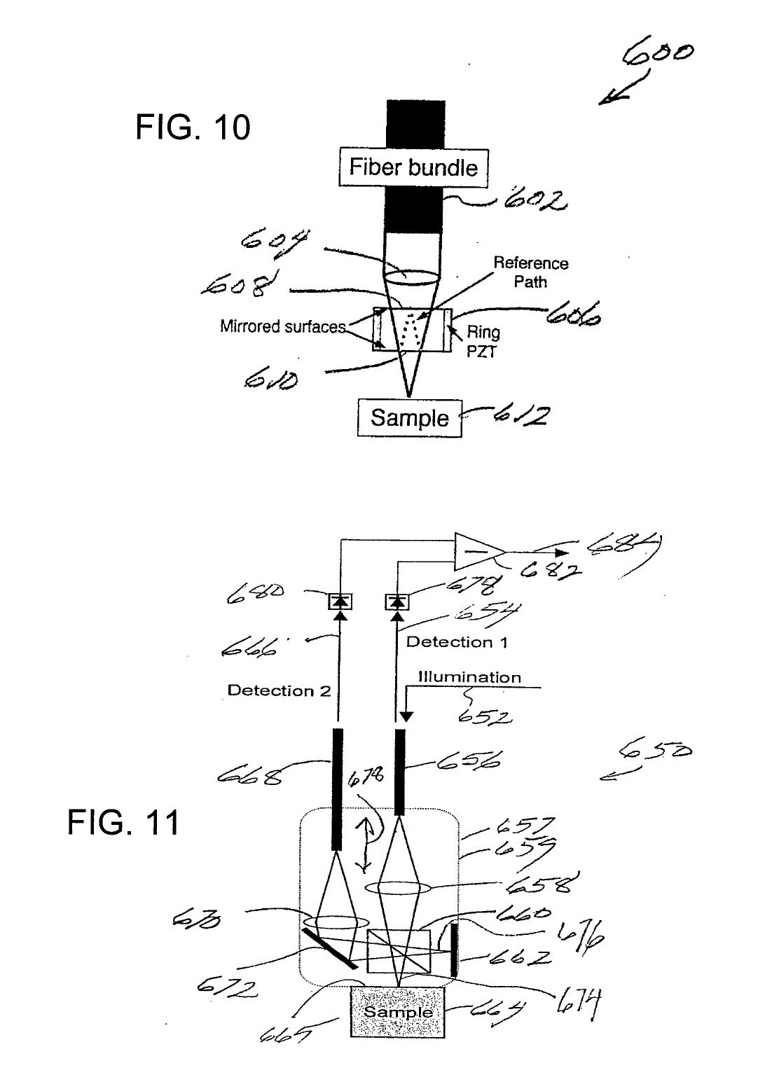

FIG. 10 shows still another exemplary embodiment of the endoscopic probe assembly 600 which can include an interferometer having a Mirau configuration. The exemplary endoscopic probe assembly 600 can be used with the single fiber-optic bundle arrangement 602 shown in FIG. 4. The exemplary probe assembly 600 can include a fixed lens 604, and a piezoelectric (PZT) ring 606 having mirrored surfaces 608, 610. The mirrored surfaces 608, 610 can provide a reference arm and a sample arm, which may direct light at a sample 612.

In the exemplary Mirau configuration, the reference path can be in-line with the sample path. The PZT ring 606 within an etalon may be actuated (e.g., changes diameter) to modify a phase difference between the reference and sample paths. The mirrored surfaces 608, 610 of the etalon may be separated by water, air, or alternatively an electro-optic crystal (e.g. BBO, LiNBO3). Certain advantages of this exemplary configuration can include compactness and stability.

FIG. 11 shows still another exemplary embodiment of the forward-looking endoscopic probe assembly 650 which can be used with a two fiber-optic bundle arrangement 656, 666, similar to the exemplary arrangement of FIG. 3. In this exemplary arrangement, as compared to the exemplary arrangement of FIG. 3, a first fiber bundle 656 can be used both for illumination and for detection, while a second fiber-optic bundle 668 may be used only for detection. The exemplary probe assembly 650 can include a probe 657 having a sheath 659 with a window 665, a fixed lens 658, another fixed lens 670, a cube-type beam splitter 660, and a mirror 662. The probe 657 may be coupled to the first fiber-optic bundle 654 and to the second fiber-optic bundle 668. A sample arm 674 may be disposed along an axis 678 of the probe 657 and a reference arm 676 can be disposed perpendicular to the axis 678 of the probe 657.

The exemplary probe assembly 650 can have a dual-balanced detection configuration. In operation, a reflected and a transmitted interference signal from the interferometer may bee detected by different detectors 678, 680 through different fiber bundles 656, 668, respectively. Because there is p phase difference between reflected and transmitted signal from the interferometer, the interference is preferably coherent. An image signal 684 can be generated by subtracting signals from the detectors 678, 680, for example, with a differencing amplifier 682.

The light detector (for example, the light detector 202 of FIG. 5) can be provided as a two-dimensional CCD camera. However, in other exemplary embodiments, the light detector can be a one-dimensional linear CCD, photodiode array, or a single light detector, e.g., elements 678, 680 of FIG. 11.

For the detection of visible light, a detection material of the light detector can be Silicon responsive to visible light (e.g., wavelength of about 0.3-1.1 μm). For the detection of near infrared light, a detection material of the light detector can be InGaAs responsive to the near infrared light (e.g., wavelength of about 1.1-2.5 μm). Exemplary features of the light detector, which can provide improved signal to noise ratio, may include a large full well depth and a high frame rate. In certain exemplary embodiments, the reference arm can be adjusted to fill half of the full well depth of the detector in order to assure shot noise limited detection.

Image reconstruction can be accomplished by, e.g., obtaining images at each of 4 positions (quadrature modulation) of the reference arm, S1=0+a, S2=p/2+a, S3=p+a, and S4=3p/2+a. The positions may be determined, for example, by the PZT stack 226 of FIG. 5. A final image can be generated using the following equation:

I=[−S₁+S₂+S₃−S₄]²+[−S₁+S₂−S₃+S₄]². (1)

The above-described arrangement generally uses a motion of the reference arm mirror (e.g., element 224 of FIG. 5) in order to impart a phase difference between reference and sample arm paths (e.g., elements 230, 228 of FIG. 5). Multiple images would be used to obtain a time-domain signal, which contains the interference information utilized for coherence gating and optical sectioning. This mode of detection is similar in general concept to time-domain OCT (“TD-OCT”).

According to another exemplary embodiment, the reference arm mirror can be fixed in position, and images may be instead acquired at different wavelengths to reconstruct the interference fringes. This mode of detection is similar in general concept to spectral-domain OCT (“SD-OCT”). When the wavelengths are simultaneously acquired, this form of coherence gating may provide an improved signal to noise ratio (“SNR”) as compared to TD-OCT. Images generated at different wavelengths may be acquired using an image spectrometer in the wavelength (e.g., frequency) or Fourier domain.

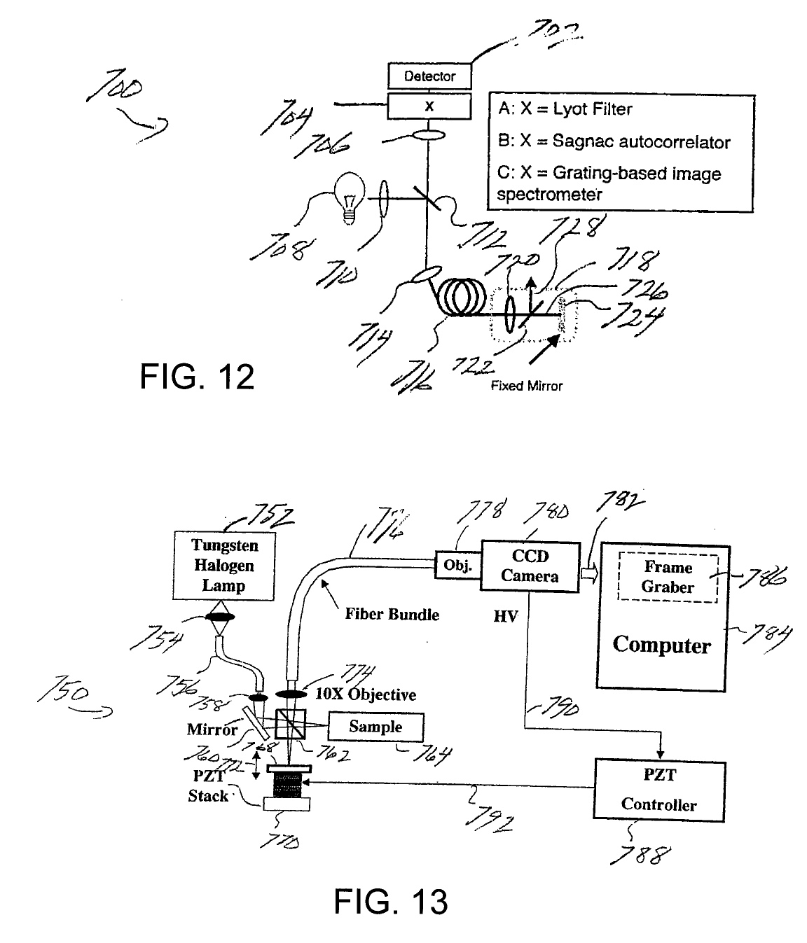

FIG. 12 shows yet another exemplary embodiment of the E-FFOCM system 700 according to the present invention, which can operates in the above-described wavelength domain, and that includes a light detector 702, a light filter 704, a lens 706, a light source 708, a lens 710, and a partially reflecting mirror 712. The exemplary E-FFOCM system 700 can also include a lens 714, a fiber-optic bundle 716, and a probe 718. The probe 718 can include a lens 720, another partially reflecting mirror 722, and a reference mirror 724. The probe 718 may include a reference arm 726 and a sample arm 728. The sample arm 728 can transmit light toward s sample (not shown).

In one exemplary embodiment, the light filter 704 can be a Lyot filter, which may be utilized to extract each individual wavelength image. In another exemplary embodiment, the light filter 704 can be a Sagnac autocorrelator. In still another exemplary embodiment, the light filter 704 may be a grating-based image spectrometer. The Sagnac autocorrelator can be utilized to obtain an autocorrelation function at the fiber bundle face and reconstruct the coherence-gated image. The grating-based image spectrometer can decompose the wavelength information at the detector 702 in a direction perpendicular to the one-dimensional fiber bundle array.

FIG. 13 shows a further exemplary embodiment of the E-FFOCM system 750 according to the present invention which can include a light source 752 (e.g., a tungsten halogen lamp). The exemplary E-FFOCM system 750 may also include a lens 754, an optical fiber 756, another lens 758, a mirror 760, a cub-type beam splitter 762, another mirror 768, and a linear actuator 770, for example, a piezoelectric (PZT) stack. The exemplary E-FFOCM system 750 can still further include yet another lens 774, a fiber-optic bundle 776, and a CCD camera 780 having an objective lens 778. The CCD camera 780 provides images 782 to a computer 784, having a frame grabber module 786.

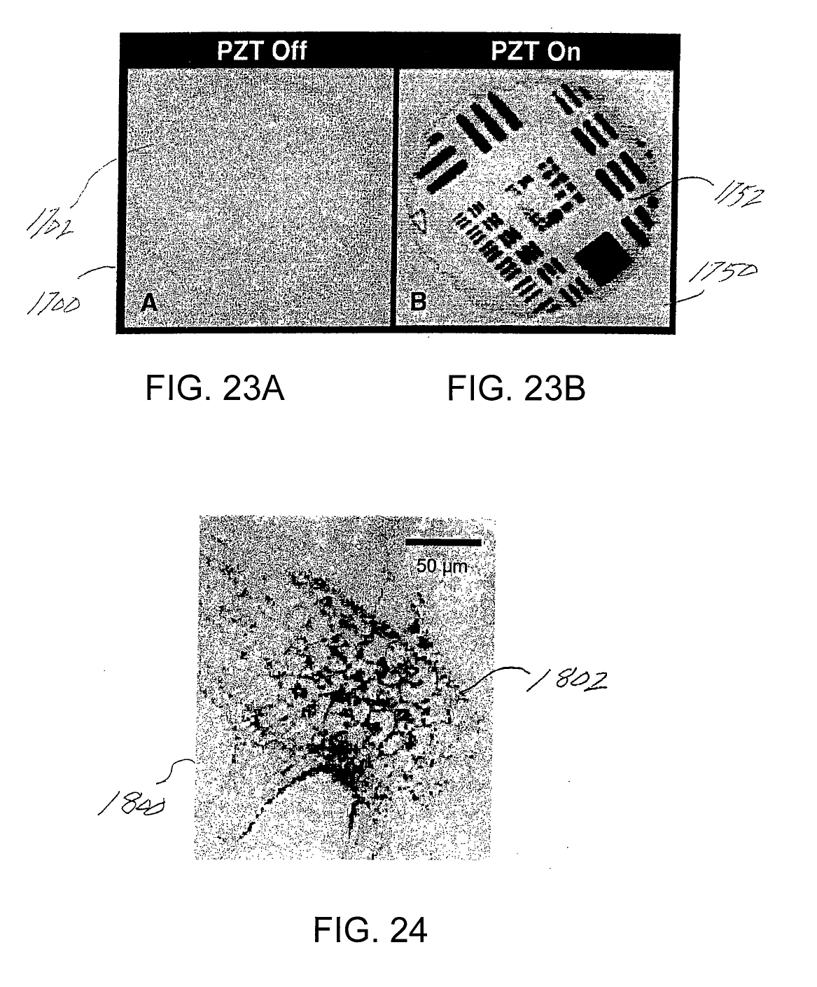

In operation, the light can be directed toward a sample 764. A PZT controller 788 may receive a frame information signal 790 from the CCD camera 780 and generate a control signal 792 to control the PZT stack, e.g., to control movement of the mirror 772 along an axis 772, in accordance with the frame information signal 790. The exemplary images generated by the exemplary system 750 of FIG. 13 are described below, and shown in FIGS. 23A and 23B.

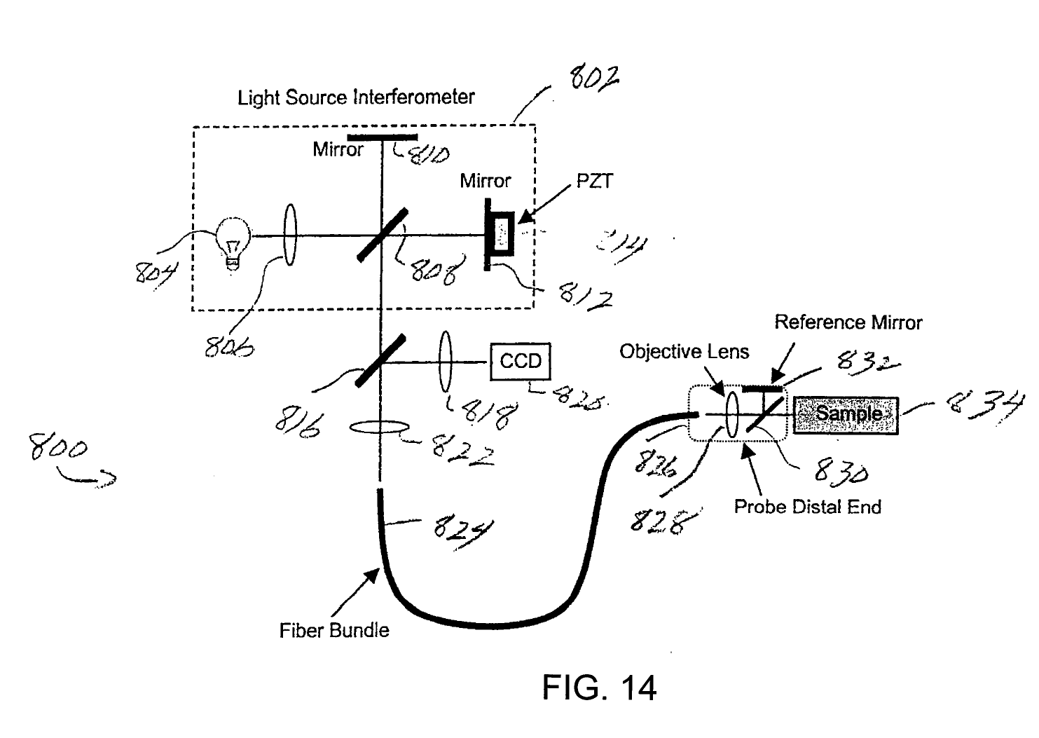

FIG. 14 shows yet further exemplary embodiment of the E-FFOCM system 800 according to the present invention, which can use a Michelson interferometer associated with a light source interferometer 802 to avoid a moving reference mirror within an endoscopic probe. The exemplary E-FFOCM system 800 can include the light source interferometer 802, which can comprise a light source 804, a lens 806, a partially reflecting mirror 808, a mirror 810, and another mirror 812. The mirror 812 can be coupled to a linear actuator 814, for example, a PZT stack. The light source interferometer 810 may form a Michelson interferometer light source.

The exemplary E-FFOCM system 800 can also include a light detector, e.g., a CCD camera 820. The exemplary E-FFOCM system 800 can still further include a lens 818, a partially reflecting mirror 816, a lens 822, a fiber-optic bundle 824, and a probe 826. The probe 826 can include a lens 828, a partially reflecting mirror 830, and a reference mirror 832.

In operation, the light can be directed toward a sample 834. Two arms of the light source interferometer 802 (e.g., the Michelson interferometer) may be adjusted so that their path length delay is identical to that of distal end interferometer within the probe 826. Multiple images for image reconstruction can be obtained at different locations of the moving reference mirror 812 of the light source interferometer 802 over one wavelength.

Using the above-described exemplary arrangement having the light source interferometer 802, the endoscopic probe 826 for OCM imaging, a moving reference mirror is not needed. As a result, the probe 826 can have a less complicated design, may be more robust, and would not require an electrical current within the probe 826. Because the probe 826 does not need a moving reference mirror, the reference mirror 832 can be placed either at the front or the side part of the probe 826.

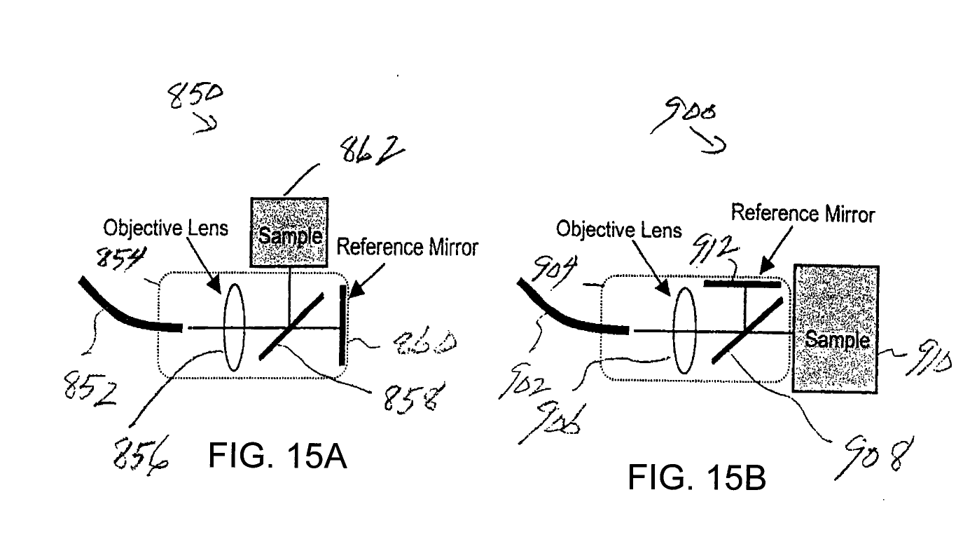

FIG. 15A shows another exemplary embodiment of the endoscopic probe assembly 850 which can include a fiber-optic bundle 852 and a probe 854. The probe 854 can comprise a lens 856, a partially reflecting mirror 858, and a reference mirror 860. The probe 852 can direct light onto a sample 862 to the side of the probe 852. FIG. 15B shows still another exemplary embodiment of the endoscopic probe assembly 900 which can include a fiber-optic bundle 902 and a probe 904. The probe 904 can comprise a lens 906, a partially reflecting mirror 908, and a reference mirror 912. The probe 904 may direct light onto a sample 910 to the end of the probe 904.

The flexibility of position of the reference mirrors 860, 912 of FIGS. 15A and 15B, respectively, can facilitate the endoscopic probe 852, 904 to support both side-looking and forward-looking configurations. In addition, since the reference mirrors 860, 912 are stationary in the exemplary respective arrangements of FIGS. 15A and 15B, as described above, a reflective facet of a beam splitter can be used in place of each of the reference mirrors 860, 912, possibly having length matching and proper coating for proper reference arm reflectivity.

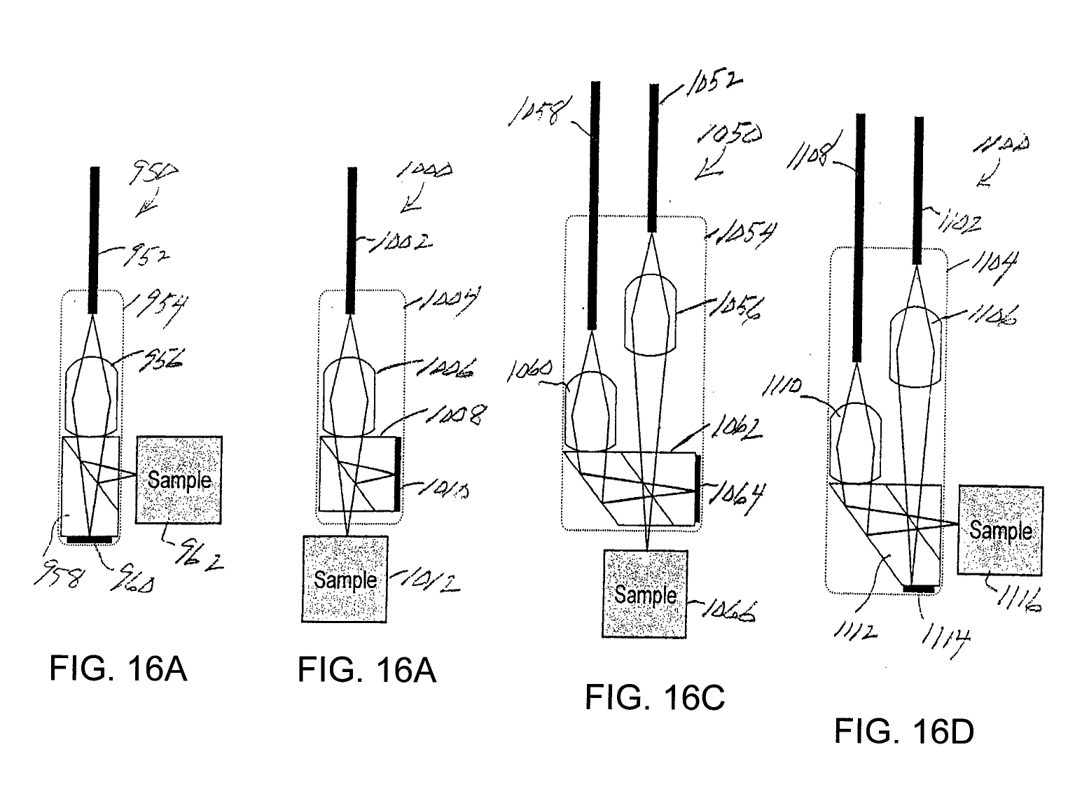

FIGS. 16A-16D show further exemplary embodiments of the endoscopic probe assemblies 950, 1000, 1050, and 1110, each possibly having different monolithic design options, and using a respective beam splitter 958, 1008, 1062, 1112 having a respective reference reflector 960, 1010, 1064, 1114 on a facet of the beam splitter.

For the reconstruction of images generated by light source interferometer modulation, several different modulation schemes can be used. For example, two images with p phase shift can be generated. In another exemplary embodiment, four images with quadrature (p/2) modulation can be generated. In still other exemplary embodiments, modulation schemes can be used having more than four images or fewer than four images at phase shifts at less than p/2 or greater than p/2. These exemplary modulations can also apply to the exemplary embodiments for which the modulation may be performed in respect to the probe reference arm length.

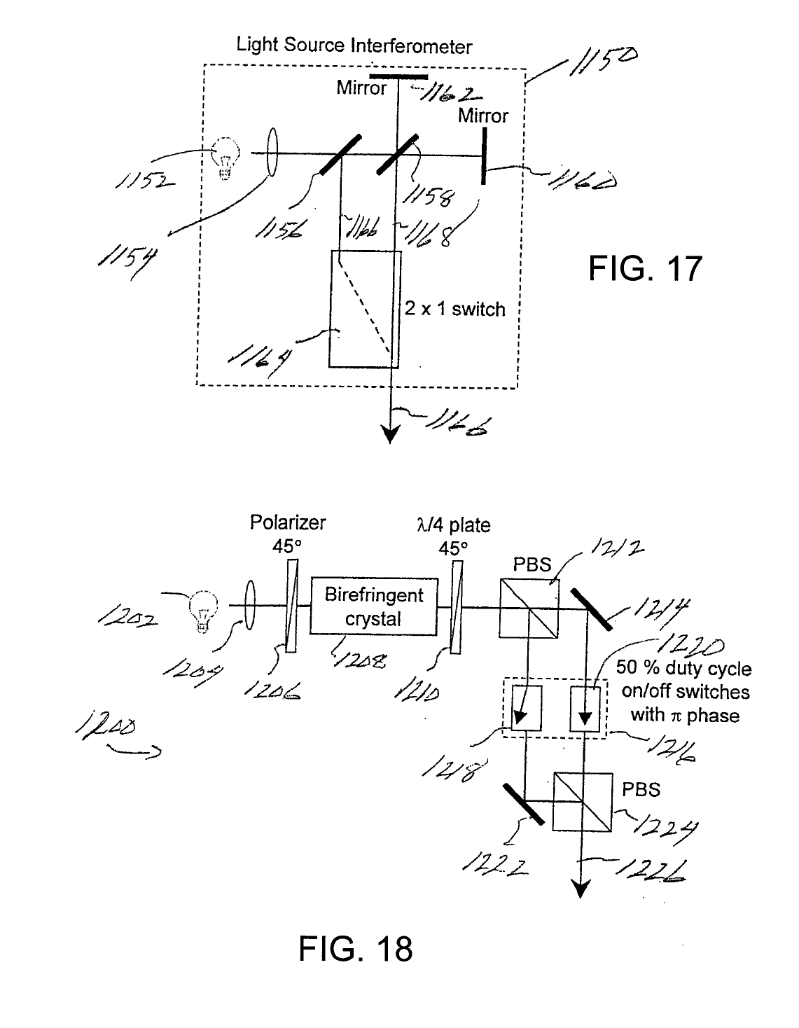

FIG. 17 shows an exemplary embodiment of a light source interferometer 1150 which can be used, for example, in place of the light source interferometer 802 of FIG. 14. The exemplary light source interferometer 1150 can include a light source 1152, a lens 1154, a partially reflecting mirror 1156, another partially reflecting mirror, 1158, a mirror 1160, a mirror 1162, and a 2×1 optical switch 1164. In operation, the reflected light can emerge from a reflected light port 1166, and transmission light emerges from a transmission port 1168.

The light emerging from the reflection port 1166 can travel a different path length that light emerging from the transmission port 1168. The arms of the light source interferometer 1150 may be stationary, and the light from the reflection port 1166 and the transmission port 1168 can be time multiplexed with the 2×1 optical switch 1164. Thus, the reflected light and transmitted light emerging from the light source interferometer 1150 can be spectrally modulated according to the above path length difference, with a p phase difference. Both the reflection port 1166 and the transmission port 1168 of the light source interferometer 1150 can be utilized for image construction. To this end, the images obtained with reflected light and transmitted light can be subtracted from each other to construct coherent images.

FIG. 18 shows another exemplary embodiment of the light source interferometer 1200, which can be used, e.g., instead of the light source interferometer 802 of FIG. 14. The exemplary light source interferometer 1200 can include a light source 1202, a lens 1204, a polarizer 1206, a birefringent crystal 1208, a quarter wave plate (for example, a λ/4 plate), a cube-type beam splitter 1212, a mirror 1214, a pair of on/off light switches 1216, a mirror 1222, another cube-type beam splitter 1224. The light 1226 can emerge from the light source interferometer 1200. The exemplary light source interferometer 1200 can provide a polarization light source modulation by way of switches 1218 and 1216, while avoiding a moving reference mirror within the distal optics of an endoscopic probe. After passing through the 45° polarizer 1206, retarder (birefringence crystal) 1208, and quarter wave plate 1210, an X polarization can be spectrally modulated with a phase retardation d(=2p(nx−xy)L/1), and a Y polarization can also be modulated with the same phase retardation with p phase difference. If two images obtained with X polarization and Y polarization are subtracted from each other, a coherent gated en-face image may be obtained from a depth corresponding to the path length delay, z=(nx−xy)L/nprobe, in a distal probe.

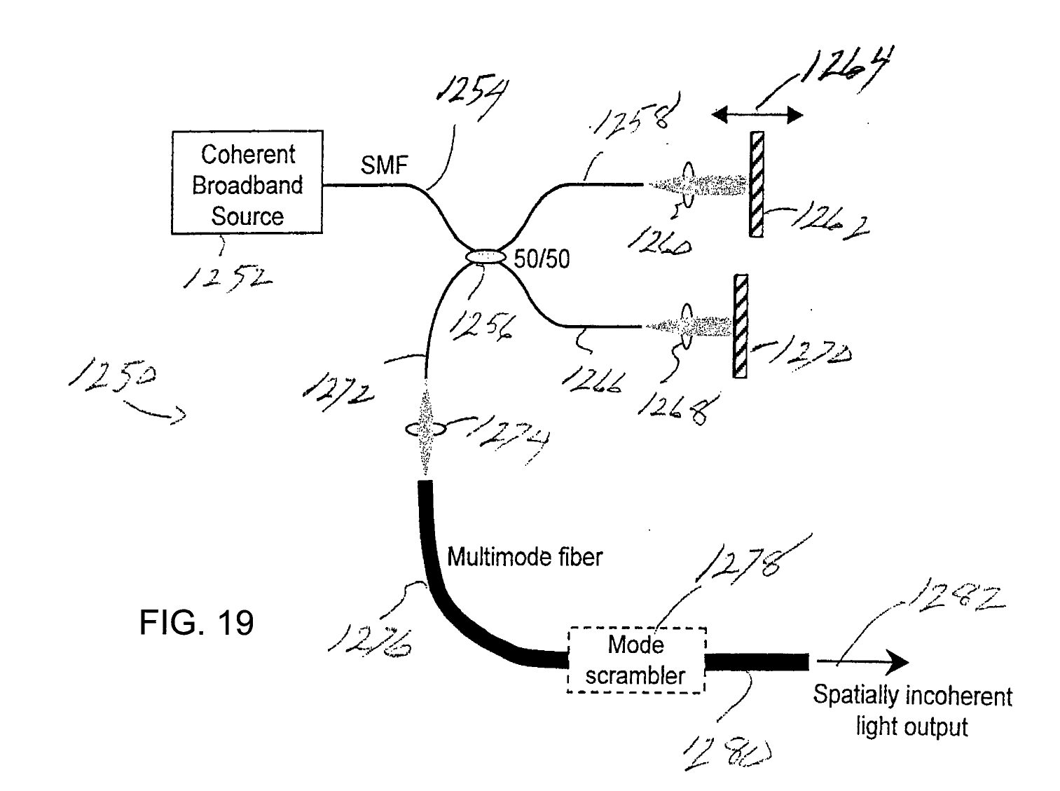

FIG. 19 shows still another exemplary embodiment of the light source interferometer 1250 (e.g., arranged as a Michelson interferometer) which can be used, e.g., instead of the exemplary light source interferometer 802 of FIG. 14. The exemplary light source interferometer 1250 can include a coherent broadband light source 1252, a single mode optical fiber 1254, a light splitter 1256, another single mode fiber 1258, a lens 1260, a mirror 1262, another mirror 1270, another lens 1268, another single mode optical fiber 1266, another single mode optical fiber 1272, another lens 1274, a multi-mode optical fiber 1276, a optional mode scrambler 1278, and another multi-mode optical fiber 1280. The coherent broadband source 1252 can include a plurality of coherent light sources, e.g. lasers, such as semiconductor laser diodes (SLDs). With the coherent light source 1252, the optical fibers 1254, 1258, 1266, 1272 can be used in the exemplary light source interferometer 1250.

For example, a better visibility of an interference signal may be obtained by using single mode light. By generating a light output from the single mode fiber (SMF) 1272, which can be coupled to the multi mode fiber (MMF) 1276 via the lens 1274, spatially incoherent light 1282 may be obtained. The spectrally incoherent light 1282 can reduce the speckle noise in the image compared with an image generated with spectrally coherent light. The mode scrambler 1278 is optional, and can be used to help multi mode excitation of the multi-mode optical fiber 1280.

FIG. 20 shows another exemplary embodiment of the E-FFOCM system 1300 which has a configuration similar to that of the exemplary E-FFOCM system 800 of FIG. 14. Similarly to the exemplary E-FFOCM system 800 of FIG. 14, the exemplary E-FFOCM system 1300 of FIG. 20 can use a Michelson interferometer associated with a light source interferometer 1302 to avoid a moving reference mirror within an endoscopic probe. The exemplary E-FFOCM system 1300 can include the light source interferometer 1302, which can comprise a light source 1302, a lens 1306, a partially reflecting mirror 1308, a movable mirror 1314, and another movable mirror 1310. The mirror 1310 can be coupled to a linear actuator 1312, for example, a PZT stack. The light source interferometer 1302 forms a Michelson interferometer light source.

In operation, either or both of the mirrors 1310, 1314 can move. The mirror 1310 can move along an axis 1313 via the PZT stack 1312. The mirror 1316 can move along an axis 1316. The exemplary E-FFOCM system 1300 can also include a light detector, for example, a CCD camera 1322. The E-FFOCM system 1300 may also include a lens 1320, a partially reflecting mirror 1318, a lens 1324, a fiber-optic bundle 1326, and a probe 1328. The probe 1328 can include an objective lens 1330, a partially reflecting mirror 1332, and a reference mirror 1336. The light may be directed toward a sample 1334.

Conventional FFOCM systems generally provide en-face tomographic images without scanning across the images in a transverse direction. However, in order to obtain en-face images at different depth of the sample using the conventional FFOCM systems, either the probe or the sample needs to move in an axial direction along an axis 1338.

For exemplary applications in which sub-micron lateral resolution across an image is not essential, the objective lens 1330 having a relatively low numerical aperture can provide a few hundred microns confocal length. Using the objective lens 1330 having this range of confocal length, the axial direction image scanning can instead be obtained by scanning one of the light source interferometer arms; i.e., by translating one of the mirrors 1310, 1314 as described above. As a result, three-dimensional volumetric imaging with better than 5 mm (lateral)×1 mm (axial) resolution can be achieved without having any mechanical scanning at the endoscopic probe's distal end.

Sensitivity of the E-FFOCM systems and methods can be directly proportional to the full-well depth of the imaging camera. Full-well depth of some line-scan cameras can be more than 100 times larger than that of area-scan cameras. For the exemplary applications where the high sensitivity is important, a line-scan camera can be used with the above-described exemplary E-FFOCM systems instead of an area-scan camera. Since the line-scan camera generally provides only one-dimensional images, mechanical scanning can be used to obtain two-dimensional images. Exemplary arrangements of the mechanical scanning are described below in conjunction with FIGS. 21A-21C.

FIGS. 21A-21D show several exemplary embodiments which can provide the above-described mechanical scanning in conjunction with endoscopic probes when used with a line-scan camera.

In particular, referring to FIG. 21A, a further exemplary embodiment of the endoscopic probe assembly 1350 according to the present invention can be provided which may include a fiber-optic line array bundle 1352 and a probe 1354. The probe 1354 can include a lens 1356, a partially reflecting mirror 1358, and a mirror 1360. In order to scan, when used in conjunction with a line-scan camera, the partially reflective mirror 1358 can be scanned along an axis 1362 transversally oriented relative to a sample 1364. In operation, the fiber-optic line array bundle 1352 can provide a one-dimensional illumination of the sample 1364, and collect and transmit the reflected light from the sample 1364 to the line-scan camera. When the partially reflecting mirror 1358 moves along the axis 1362, both lateral and depth imaging can be obtained.

FIG. 21B shows yet another exemplary embodiment of the side looking endoscopic probe assembly 1400 according to the present invention which can include a fiber-optic line array bundle 1402 and a probe 1404 having an inner assembly 1406. The inner assembly 1406 can include a lens 1408, a partially reflecting mirror 1410, and a mirror 1412. In order to scan, when used in conjunction with a line-scan camera, the inner assembly 1406 can be scanned along an axis 1416 transversally oriented relative to a sample 1414. In operation, the fiber-optic line array bundle 1402 can provide a one-dimensional illumination of the sample 1414, and collect and transmit the reflected light from the sample 1414 to the line-scan camera. When the inner assembly 1406 moves along the axis 1416, both lateral and depth imaging can be obtained.

FIG. 21C shows an exemplary embodiment of the forward looking endoscopic probe assembly 1450 according to the present invention which can include a fiber-optic line array bundle 1452 and a probe 1454 having an inner assembly 1456. The inner assembly 1456 can include a lens 1458, a partially reflecting mirror 1460, and a mirror 1462. In order to scan, when used in conjunction with a line-scan camera, the inner assembly 1456 can be scanned along an axis 1466 perpendicularly oriented relative to a sample 1414. In operation, the fiber-optic line array bundle 1452 can provide a one-dimensional illumination of the sample 1464, and collect and transmit the reflected light from the sample 1464 to the line-scan camera. When the inner assembly 1456 moves along the axis 1466, both lateral and depth imaging can be obtained.

In other exemplary embodiment of the arrangements according to the present invention, however, two-dimensional cross-section images can be obtained while using a line scan camera, without any moving parts in the endoscopic probe, by scanning one of the light source interferometer arms when sub-micron lateral resolution is not required.

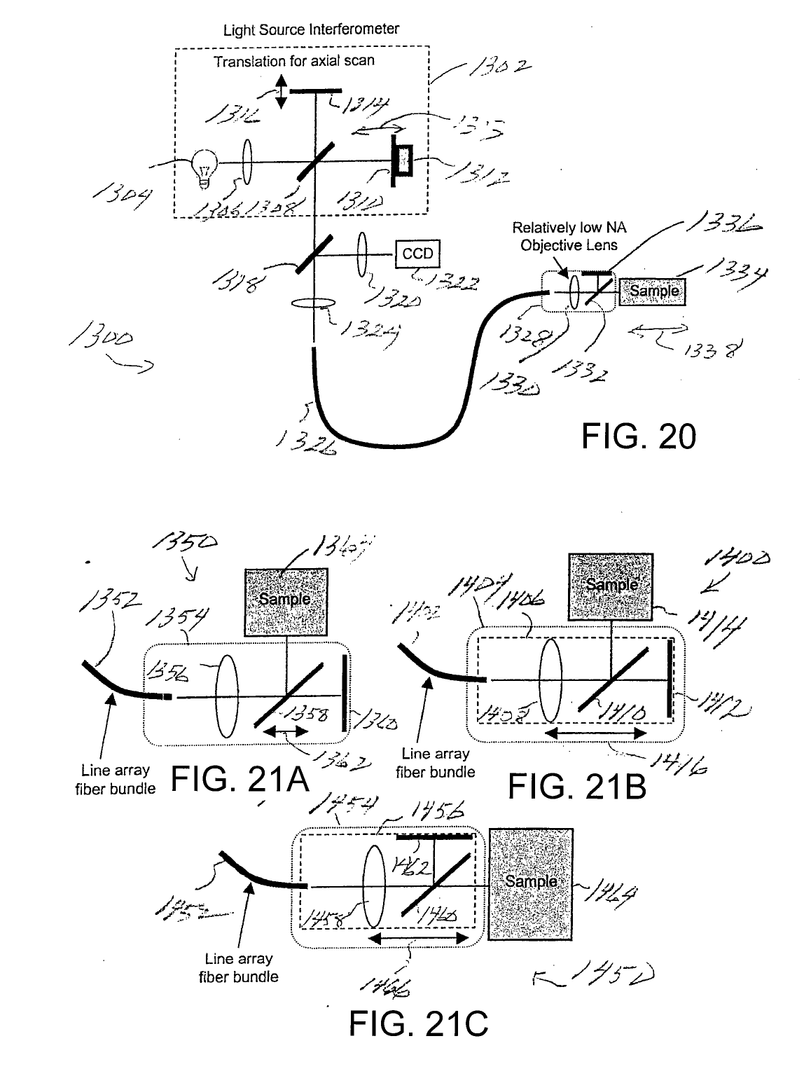

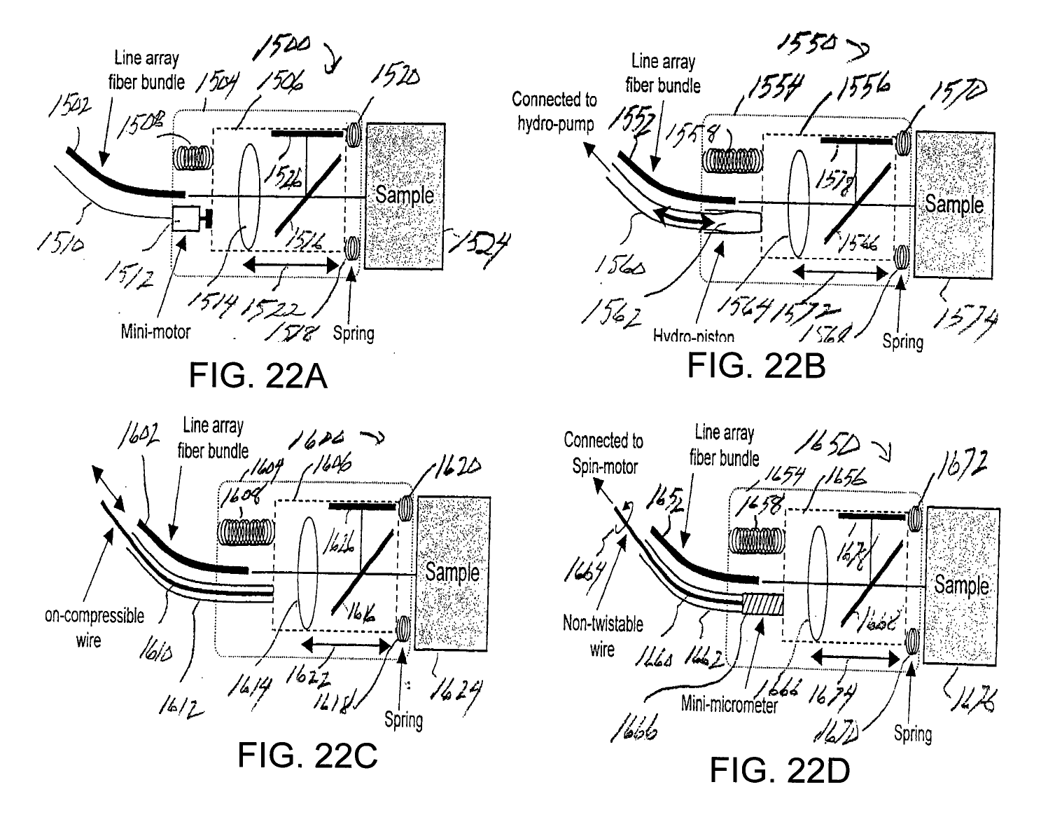

FIGS. 22A-22D show further exemplary embodiments of the configurations according to the present invention can be used to achieve the scanning described above in conjunction with the exemplary system of FIG. 21C.

In particular, FIG. 22A shows a first particular exemplary embodiment of the endoscopic probe assembly 1500 according to the present invention which can include a fiber-optic bundle 1502 and a probe 1504 with an inner assembly 1506. The inner assembly 1506 can include a lens 1514, a partially reflecting mirror 1516, and a mirror 1526. The inner assembly 1506 can be suspended by springs 1508, 1518, 1520. A miniature translation motor 1512 can move the inner assembly 1506 along an axis 1522 in order to scan a sample 1524.

FIG. 22B shows a second particular exemplary embodiment of the endoscopic probe assembly 1550 according to the present invention which can include a fiber-optic bundle 1552 and a probe 1554 with an inner assembly 1556. The inner assembly 1556 can include a lens 1564, a partially reflecting mirror 1566, and a mirror 1578. The inner assembly 1556 can be suspended by springs 1558, 1568, 1570. A hydraulic or pneumatic piston 1562 coupled by a tube 1560 to a pump (not shown) can move the inner assembly 1556 along an axis 1572 in order to scan a sample 1574.

FIG. 22C shows a third particular exemplary embodiment of the endoscopic probe assembly 1600 according to the present invention which can include a fiber-optic bundle 1602 and a probe 1604 with an inner assembly 1606. The inner assembly 1606 can include a lens 1614, a partially reflecting mirror 1616, and a mirror 1626. The inner assembly 1616 can be suspended by springs 1608, 1618, 1620. A wire 1610, linearly movable within a sleeve 1610, can move the inner assembly 1616 along an axis 1622 in order to scan a sample 1624.

FIG. 22D shows a fourth particular exemplary embodiment of the endoscopic probe assembly 1650 according to the present invention which can include a fiber-optic bundle 1652 and a probe 1654 with an inner assembly 1656. The inner assembly 1656 can include a lens 1666, a partially reflecting mirror 1668, and a mirror 1678. The inner assembly 1656 can be suspended by springs 1658, 1670, 1672. A wire 1660, rotationally movable within a sleeve 1662 can move screw type micrometer 1666 to move the inner assembly 1656 along an axis 1674 in order to scan a sample 1676.

Referring to the exemplary images of FIGS. 23A and 23B, exemplary images 1700, 1750 of a 1951 US Air Force resolution chart 1702, 1752 have been obtained using the exemplary system of FIG. 13. For the image shown in FIG. 23A, the PZT linear actuator 770 (shown in FIG. 13) was turned off, and for the image shown in FIG. 23B, the PZT linear actuator 770 was operating. The exemplary images 1700, 1750 were obtained through a 160 μm thick 1% intralipid solution, which is equivalent to imaging through 160 μm of human tissue. Image quality was found to be independent of orientation of the fiber-optic bundle 776 (FIG. 13) as is desired. These exemplary images have lower speckle noise due to use of a multi-mode fiber-optic bundle 776.

FIG. 24 shows an exemplary en-face sectional image 1800 of the African frog tadpole Xenopus laevis, which was obtained ex vivo, 200 mm below the surface using the exemplary embodiment of the FFOCM system which includes a Michelson light source interferometer. Cell walls and nuclei 1802 are shown in this exemplary image, demonstrating the high resolution of E-FFOCM.

The foregoing merely illustrates the principles of the invention. Various modifications and alterations to the described embodiments will be apparent to those skilled in the art in view of the teachings herein. Indeed, the arrangements, systems and methods according to the exemplary embodiments of the present invention can be used with and/or implement any OCT system, OFDI system, SD-OCT system or other imaging systems, and for example with those described in International Patent Application PCT/US2004/029148, filed Sep. 8, 2004, U.S. patent application Ser. No. 11/266,779, filed Nov. 2, 2005, and U.S. patent application Ser. No. 10/501,276, filed Jul. 9, 2004, the disclosures of which are incorporated by reference herein in their entireties. It will thus be appreciated that those skilled in the art will be able to devise numerous systems, arrangements and methods which, although not explicitly shown or described herein, embody the principles of the invention and are thus within the spirit and scope of the present invention. In addition, to the extent that the prior art knowledge has not been explicitly incorporated by reference herein above, it is explicitly being incorporated herein in its entirety. All publications referenced herein above are incorporated herein by reference in their entireties.

What is claimed is:

1. A system for imaging at least one portion of a sample, comprising:
at least one first arrangement configured to receive at least one first electro-magnetic radiation from the sample and to receive at least one second electro-magnetic radiation from a reference, wherein the at least one first arrangement and the reference are provided in an endoscope enclosure; and
at least one second detection arrangement configured to generate image data associated with the at least one portion as a function of the first and second electro-magnetic radiations.

2. The system according to claim 1, further comprising at least one third arrangement being in communication with the at least one first arrangement and configured to receive at least one third electro-magnetic radiation from a further reference, wherein the at least one third arrangement is provided outside of an endoscope enclosure.

3. The system according to claim 2, wherein the further reference is a translatable reference, and wherein the at least one third arrangement is further configured to receive at least one fourth electro-magnetic radiation from a stationary reference, wherein the translatable and stationary references are provided externally from the endoscope enclosure.

4. The system according to claim 3, further comprising a fourth arrangement configured to move the translatable reference.

5. The system according to claim 4, wherein the fourth arrangement is a piezo-electric transducer.

6. The system according to claim 2, wherein the at least one first arrangement communicates with the at least one third arrangement via a fiber arrangement.

7. The system according to claim 6, wherein the fiber arrangement includes at least one of a single fiber or a plurality of fibers.

8. The system according to claim 6, wherein the fiber arrangement is at least one of a single model arrangement or a multi-mode arrangement.

9. The system according to claim 6, wherein a first fiber of the fiber arrangement is configured to transmit an electro-magnetic radiation to the sample, and wherein the first fiber and a second fiber of the fiber arrangement is configured to receive the at least one first electro-magnetic radiation from the sample and the at least one second electro-magnetic radiation from the reference.

10. The system according to claim 9, wherein the first and second fibers transmit a further electro-magnetic radiation for performing a dual balance detection.

11. The system according to claim 2, wherein the further reference is fixed, wherein the at least one third arrangement comprises a beam splitting arrangement providing a fourth electro-magnetic radiation and a fifth electro-magnetic radiation that are out of phases from one another.

12. The system according to claim 11, further comprising at least one fourth arrangement which selectively forwards at least one of the fourth or fifth electro-magnetic radiations to the at least one first arrangement.

13. The system according to claim 12, wherein the at least one fourth arrangement is an optical switch.

14. The system according to claim 1, wherein the at least one first arrangement is an interferometric arrangement.

15. The system according to claim 14, wherein the interferometric arrangement comprises at least one of a Michelson interferometer, a Linnik interferometer, a Mach-Zehnder interferometer, a common path interferometer, a Sagnac interferometer or a Mirau interferometer.

16. The system according to claim 14, wherein the interferometric arrangement is monolithic.

17. The system according to claim 1, wherein the reference includes an attenuator.

18. The system according to claim 1, wherein the reference is translatable.

19. The system according to claim 1, wherein the system is part of an endoscopic arrangement, and wherein the second arrangement situated within and at one end of an endoscope enclosure of the endoscope arrangement.

20. The system according to claim 1, wherein the second arrangement is at least one Linnik interferometric arrangement.

21. An endoscope arrangement for imaging at least one portion of a sample, comprising:
at least one interferometric arrangement configured to receive at least one electro-magnetic radiation from the sample, and situated within and at one end of an endoscope enclosure of the endoscope arrangement.

22. The endoscope arrangement according to claim 21, wherein the one end of the endoscope enclosure is provided in a proximity of the sample.

23. The endoscope arrangement according to claim 21, wherein the at least one interferometric arrangement is a Linnik interferometric arrangement.

24. The endoscope arrangement according to claim 21, wherein the at least one interferometric arrangement is immersed in a fluid.

25. The endoscope arrangement according to claim 21, wherein the at least one interferometric arrangement comprises a beam splitting arrangement providing a first further electro-magnetic radiation and a second further electro-magnetic radiation that are out of phases from one another.

26. The endoscope arrangement according to claim 25, further comprising at least one further arrangement which selectively forwards at least one of the first or second further electro-magnetic radiations to at least one fiber arrangement.

27. The endoscope arrangement according to claim 26, wherein the at least one third arrangement is at least one of an optical switch or a plurality of fibers.

28. A system for imaging at least one portion of a sample, comprising:
at least one first Linnik interferometric arrangement; and
at least one second fiber arrangement being in optical communication with the at least one first arrangement,
wherein at least one second arrangement is configured to transmit at least one first electro-magnetic radiation to the at least one first arrangement; and
wherein the at least one first arrangement is configured to receive at least one second electro-magnetic radiation from the sample which is associated with the at least one first electro-magnetic radiation, and
wherein the at least one first arrangement is configured to forward at least one third electro-magnetic radiation which is associated with the at least one second electro-magnetic radiation to the at least one second arrangement.

29. The system according to claim 28, wherein the at least one second arrangement is configured to transmit imaged data associated with the at least one portion.

30. The system according to claim 28, further comprising at least one third arrangement configured to receive the image data, and generate at least one image of the at least one portion based on the image data.

31. The system according to claim 28, wherein the at least one second arrangement is a fiber bundle.

32. The system according to claim 28, wherein at least one first fiber of the at least one second arrangement is configured to transmit the at least one first electro-magnetic radiation, and wherein at least one second fiber of the at least one second arrangement is configured to transmit the at least one third electro-magnetic radiation.

33. The system according to claim 28, wherein at least one fiber of the at least one second arrangement is configured to transmit the at least one first electro-magnetic radiation and the at least one third electro-magnetic radiation.

34. The system according to claim 28, wherein the first and second arrangements are provided in a catheter enclosure or in an endoscope enclosure.

35. The system according to claim 28, wherein the at least one first interferometric arrangement is immersed in a fluid.

36. The system according to claim 28, wherein the at least one first arrangement comprises a beam splitting arrangement providing the at least one third electro-magnetic radiation and a fourth electro-magnetic radiation that are out of phases from one another.

37. The system according to claim 36, further comprising at least one third arrangement which selectively forwards at least one of the third or fourth further electro-magnetic radiations to the at least one second arrangement.

38. The system according to claim 37, wherein the at least one third arrangement is at least one of an optical switch or a plurality of fibers.

39. A method for imaging at least one portion of a sample, comprising:
receiving at least one first electro-magnetic radiation from the sample and at least one second electro-magnetic radiation from a reference using at least one arrangement, wherein the at least one arrangement and the reference are provided in an endoscope enclosure; and
generating image data associated with the at least one portion as a function of the first and second electro-magnetic radiations.
